(19) United States
(12) Patent Application Publication
(10) Pub. No.: US 2004/0053354 A1
(43) Pub. Date: Mar. 18, 2004

(54) Methods for optically immobilizing very small objects and their use

(75) Inventors: Taiji Ikawa, Aichi-gun (JP); Osamu Watanabe, Nagoya-shi (JP); Fumihiko Hoshino, Aichi-gun (JP); Takashi Matsuyama, Aichi-gun (JP); Tsutomu Kajino, Toyoake-shi (JP); Haruo Takahashi, Ohgaki-shi (JP); Masaaki Tsuchimori, Owariasahi-shi (JP); Takuya Mitsuoka, Nishikamo-gun (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOYOTA CHUO KENKYUSHO, Aichi-gun (JP)

(21) Appl. No.: 10/377,799

(22) Filed: Mar. 4, 2003

(30) Foreign Application Priority Data
Mar. 4, 2002 (JP) 2002-056752
Feb. 17, 2003 (JP) 2003-038039

(51) Int. Cl. G01N 1/30; G01N 33/48

(52) U.S. Cl. 435/40.5

(57) ABSTRACT

A method for optically immobilizing very small objects, where a material with the capability of photoinduced deformation to possibly immobilize very small objects is used at least as a surface layer of a carrier and where the very small objects are immobilized via irradiation light while they are arranged on the surface of the carrier. Very small objects particularly preferably include proteins, nucleic acids and the like. A very small object-immobilized carrier having immobilized very small objects in such manner, particularly a biosensor. A method for observing a very small object immobilized on the surface of a carrier by an appropriate approach giving displacement force to the very small object. The present invention provides a method for strongly immobilizing very small objects on the surface of a carrier with a simple tool.

### BACKGROUND OF THE INVENTION

1. Field of the Invention

The present invention relates to a method for optically immobilizing very small objects, a carrier immobilizing very small objects thereon and a method for observing very small objects. More specifically, the invention relates to a useful and absolutely novel tool for immobilizing very small objects, namely for physically immobilizing various very small objects on a carrier, using optical means.

2. Description of the Related Art

In many technical fields of material and mechanical sciences and the like, recently, nanotechnology for subject analysis or fabrication at an extremely small scale has been drawing attention. In the field of biotechnology, additionally, fruitful research results from the fusion of the nanotechnology in the above fields with molecular biology has been drawing attention as well.

A technique for immobilizing for example very small nanometer-scale metal particles, metal oxide particles, semiconductor particles, ceramics particles, plastic particles or complex particles thereof with given functions on an appropriate carrier or substrate has been desired. Furthermore, a technique for aligning and immobilizing numerous such particles in a predetermined pattern has also been desired.

Following the distinct progress made in molecular biology, specifically the progress or completion of various genomics projects, for example, attention is now focused on the functional analysis of functional biomolecules and the like. Such functional biomolecules include for example genes, enzymes expressed by genes, antigen-antibody of importance in immunoassay and cell membrane receptor-protein responsible for biological signal transduction. For these relations, in vitro analysis using cells and microorganisms is also very important.

Furthermore, one of dominating issues in the medical field since the genomics projects is gene diagnosis or DNA diagnosis. In other words, it is suggested that novel and useful pharmaceutical products can be created (so-called gene-based medicine) by the analysis of RFLP (restriction fragment length polymorphism) or DNA fragments containing microsatellite part and by the analysis of diverse single nucleotide polymorphisms (SNPs). Additionally, it is also suggested that personal gene information if got will be applied to “tailor-made” medicine or forensic identification.

In the circumstances, a technique has been desired for immobilizing very small metal particles, metal oxide particles, semiconductor particles, ceramic particles, plastic particles and the like on an appropriate carrier or substrate securely in a simple manner. More preferably, a simple technique for immobilizing these functional very small particles in a desired distribution pattern like integrated circuit chip for example has also been desired. When a technique effective for filming or immobilizing inorganic functional particles such as metal particles, metal oxide particles, and semiconductor particles on the surface of a solid is provided, the technique is useful in using these particles as catalysts for the purpose of antibacterial treatment or photodecomposition. The technique is also useful in using these particles for large-scale integration of electronic devices.

Additionally, the technique can be applied to a device controlling the refraction, separation, etc. of optical wave by arranging particles with different optical profiles in a preset cyclic structure to generate photonic bands. Additionally, dielectric particles such as silica particle have a function to entrap light. Therefore, the immobilization of these dielectric particles on optical waveguide enables laser oscillation. The technique is useful for the practical application of such laser oscillation device.

As specific examples of the technique for filming or immobilizing an inorganic functional particle on solid substrates, JP-T(TOKUHYO)-11-514755 discloses a method for immobilizing very small particles on substrates, using a curable composition. According to the method, a curable composition containing particles of a particle size of about 1 μm is applied onto a substrate. Under given conditions, the curable composition is polymerized, to form a cured film of a thickness ½-fold or less the particle size. Then, the non-cured curable composition is removed, to form a single layer of the particles on the substrate. However, the method requires a step of removing the non-cured curable composition, which complicates the production steps, and the method involves difficulty in the control of the viscosity of the curable composition and the control of the surface flatness of the film, disadvantageously.

JP-A-11-90213 discloses a method for forming an ultra thin film, including a step of coating a colloid dispersion of an inorganic particle on the surface of hydrogel to thereby form an ultra thin film of the inorganic particle and a step of putting the ultra thin film in contact to a solid substrate for transfer. However, JP-A-11-90213 never describes any method for immobilizing the ultra thin film on the solid substrate.

Nature, vol. 415, p.621 (2002) describes a technique for laser oscillation, using the function of silica particle to entrap light, including a step of putting single mode fiber in contact onto silica particle. However, the technique is not practical because the single mode fiber is just simply put in contact.

Meanwhile, protein chips immobilizing various polypeptides as functional biomolecules on carriers as well as DNA chips or DNA microarrays immobilizing genes or various DNA fragments on carriers are now going to be established as very significant research & development tools. However, these very small objects as subjects to be immobilized are very delicate. It is concerned that polypeptides might lose enzyme functions, antigen/antibody functions, the binding capacities of membrane proteins with ligands, and the like due to the modification of their configurations or chemical structures during immobilization. Polynucleotides such as DNA are also problematic in terms of the intensity of their immobilization and the mode of immobilizing such polynucleotides while they still retain their hybridization potencies.

For example, a problem is remarked such that the amount of DNA attached on poly-L-lysine-coated slide glass for general use as a carrier for DNA microarray is not constant and is at very poor reproducibility (“DNA microarray and latest PCR”, p. 127; Shujun-sha Co. Ltd., 2000).

The problem is frequently ascribed to the blocking process after DNA spotting. The term blocking process means a process of inactivating the amino group of the poly-L-lysine at positions except for the position with spotted DNA, so as to avoid adsorption of the test subject DNA at unexpected positions on a carrier. General blocking process includes a step of immersing a DNA-spotted carrier in a treatment agent (an organic solvent mixed with succinic acid) to amidate the amino group for the inactivation. During the treatment, disadvantageously, the DNA on the carrier is detached.

Additionally, carriers immobilizing biomolecules such as protein other than DNA thereon require such blocking process. In that case, disadvantages occur, including protein denaturation, complicated treatment procedures and the occurrence of error during assaying.

Following the progress of molecular biology, in recent years, it is demanded to analyze numerous proteins and nucleic acid molecules by multivariate analysis. The carrier for immobilizing biomolecules for multivariate analysis has to be able to immobilize molecules with different physical and chemical properties. By immobilization techniques so far, it has been difficult to immobilize diverse types of biomolecules.

Furthermore, a carrier immobilizing cells or microorganisms thereon and an observation means of the cells or microorganisms immobilized thereon are now needed as important means for research and development. If an observation means capable of analyzing the configuration and function of protein immobilized on a carrier can be provided, such observation means becomes a very efficacious one for research and development. Even in these cases, the procurement of cells or microorganisms at viable state and the retention of protein configuration during their immobilization are also problematic.

Techniques so far capable of coping with such technical problems include for example the following techniques.

As the technique for immobilizing enzymes on carriers, the following techniques have been known: the entrapment method of immobilization including a step of entrapping enzymes in gel; the microcapsule method of coating enzymes with semi-transparent polymer film; and the surface modification method of modifying and stabilizing the surface of enzymes with polyethylene glycol or glycolipid. However, any of these methods has drawbacks such that the structure unit for immobilizing enzymes does not have a shape to stably fix enzyme molecules (for example, a simple flat face) or that even if the structure unit has such shape, the structure unit lacks structural stability. Therefore, the configuration of immobilized enzymes cannot be retained in a stable manner.

Concerning DNA chips, a great number of techniques have been proposed, which includes a step of aligning and integrating a great number of/numerous types of single-stranded DNAs, a step of allowing the DNAs to hybridize with cDNA or genome DNA to analyze the gene expression profile at genome scale. Recently, a proposition has been made about DNA chips using DNA fragments containing SNP part or DNA fragments containing microsatellite part in place of DNA fragment as gene or DNA fragment composing a part of gene as the DNA to be aligned and integrated.

As a specific example thereof, the invention of a method for preparing DNA chip in accordance with the published specification of JP-A-11-21293 discloses a method for immobilizing a great number of DNAs as gene or composing a part of gene, including a step of aligning and integrating such DNAs. However, the method is based on photolithography. Therefore, the method requires complicated procedures such as the multi-layer preparation and etching of the substrate, and chemical reactions after circuit structures are imprinted. Thus, the production efficiency of the method is low, while the production cost is very high.

Regarding tools for observing or analyzing cells, microorganisms or enzyme proteins, scanning probe microscopy (SPM) typically including atomic force microscopy is listed. By SPM, sharp probe is used to trace the surface of a sample. When the sample is not fixed, however, the position of the sample moves following the motion of the probe. Thus, no accurate sample image can be obtained. When the sample is a living microorganism, the problem is more serious. According to a method for fixing samples with a highly viscous resin such as gel, the resin adheres to the probe, so that no accurate sample image can be recovered.

### SUMMARY OF THE INVENTION

It is an object of the invention to provide a method for immobilizing very small objects on the surface of a carrier with a simple means. It is an additional object of the invention to provide a method for immobilizing a great number of and/or numerous types of very small objects including a step of aligning such very small objects on the surface of a carrier. It is a still additional object of the invention to provide a method for immobilizing these very small objects without deterioration of the viable states thereof or of the intrinsic functions thereof. Further, it is an object of the invention to provide a carrier immobilizing very small objects in such manner thereon and a method for observing very small objects.

In a first aspect, the invention relates to a method for optically immobilizing very small objects, comprising a step of fabricating a carrier using the following material (A) for optical immobilization at least as a surface layer, a step of arranging the following very small objects (B) on the surface of the carrier, and a step of immobilizing the very small objects thus arranged on the surface of the carrier via light irradiation:

(A) the material for optical immobilization: a material with the capability of photoinduced deformation, which exerts the potency of immobilizing the very small objects arranged on the surface of the carrier during light irradiation; and

(B) the very small objects: a tangible object with a size of 50 μm or less.

In the first aspect, the term “photoinduced deformation” includes deformation in general sense and additionally includes micro-deformation due to the interaction between very small object and carrier surface (for example, membrane surface) via the movements at molecular levels. Some of such photoinduced deformations can clearly be observed with optical microscope and electron microscope, but it is difficult to clearly observe some of the photoinduced deformations with general observation tools, due to problematic deformation level and deformation type.

Very small objects can be immobilized singly and separately in some case, while in other case, numerous very small objects are immobilized, following a specific distribution pattern. Besides, a very small object with a self-assembly property is sometimes arranged and immobilized at the self-assembling state of a great number of the very small objects on the surface of a carrier.

In the course of research works about a means for solving the problems, the present inventors have found a very interesting, novel finding that optical irradiation of a very small object when arranged on the surface of a material with the capability of photoinduced deformation permits the very small object to be more intensely immobilized on the surface of the material. Currently, the reason is not essentially clear. Possibly, however, the plasticization of the surface of the material on which a very small object is arranged and the photoinduced deformation of the surface of the material, depending on the shape of the very small object (corresponding to the shape of the very small object in many cases) and the like, may have some relation.

Essentially, the material (A) for optical immobilization in the first aspect includes any material with the capability of photoinduced deformation. It is suggested that such material possibly exerts the potency of immobilizing very small objects arranged on the surface of the carrier during light irradiation. The material for optical immobilization, which can exert such effect, preferably includes for example photochromic materials capable of changing their molecular structures via photoabsorption, particularly materials with the capability of photoisomerization involving large structural changes, such as cis-trans isomerization.

The type of the very small object (B) in the first aspect is not specifically limited. Preferably, however, the very small object is of a size of 50 μm or less. When the very small object is of a size above 50 μm, potentially, it is difficult to optically immobilize the very small object sufficiently. More preferably, the very small object is of a size of 10 μm or less. With respect to the lowest limit size of the very small object, very small objects of sizes of protein molecules or nucleic acid molecules or of sizes of about 1 nm are subjects. When the very small object is smaller than these sizes (below 1 nm for example), the photoinduced deformation of a material for optical immobilization is potentially insufficient (the action for immobilizing the very small object is insufficient). Concerning the lowest limit size of the very small object, the term “size” means the size along the short diameter direction of the very small object (for DNA as a slender molecule, for example, the size means not the length but the width).

Light irradiation of a very small object arranged on the surface of a carrier using a material for optical immobilization at least as the surface layer induces an electric field around the very small object. Depending on the electric field, the material for optical immobilization deforms, depending on the shape of the very small object. Such deformation includes for example a deformation corresponding to the shape of the very small object (deformation depending on the very small object, such as deformation in the relation between molded article and mold). Consequently, the effect of the deformed carrier surface on the very small object as a support, the effect on the increase of adhesion force such as van der Waals force due to the increase of the contact area between the carrier surface and the very small object and the like can give an effective immobilization force of the very small object.

The method for optically immobilizing very small object in the first aspect has the following actions and advantages.

First, the method can be conducted by a very simple approach and at a simple process. Thus, the method can be conducted at low cost. In other words, the following three conditions are needed: a material with the capability of photoinduced deformation; the practical preparation of a state of a very small object arranged on (in contact to) the material; and optical irradiation at that state.

Second, the method is very widely applicable. For example, the type of the very small object essentially includes all hard and soft non-fluidic materials as the subjects, for example inorganic materials such as metal particles and semiconductor particles, organic materials such as plastic particles, biological molecules such as protein and DNA, cells and microorganisms, with no limitation. Additionally, a large number of each of these various types of very small objects can be immobilized on one such carrier. For example, a large number of plural types of biological molecules such as proteins of various types and diverse properties and of various sizes can be immobilized on one such carrier. Therefore, the method is preferable for the multivariate analysis of biological molecules.

Third, diverse modes for carrying out the method can be used. When the carrier and a very small object are put in contact to each other in a buffer, for example, cells or microorganisms can be immobilized at their viable states thereof. Modification of the irradiation pattern of light with an appropriate tool can modify the distribution pattern of the very small object to be immobilized.

Fourth, it is suggested that because the means for immobilization is light irradiation, the immobilization mechanism is largely dominated by physical adsorption. Compared with immobilization via chemical bonding, immobilization with binders and the like, or immobilization via the formation of an immobilizing structure, for example, the method has very small adverse effect on the function of the very small object. The method can effectively prevent for example enzyme inactivation or the deformation of cell or protein, after immobilization. Further, blocking process for the immobilization of biological molecules as in the related art is not any more needed.

It is very easy to simultaneously immobilize a great number of each of numerous diverse types of very small objects by the method for optically immobilizing a very small object. When these very small objects have a self-assembly property, the very small objects at their self-assembling state can be immobilized. Therefore, the expression of a biochemical function specific to a macromolecule protein derived from the self-assembly of numerous protein molecules is allowed at the immobilized state thereof on a carrier. Similarly, the formation of photonic band due to the two-dimensional photonic crystal structure, the formation of three-dimensional photonic crystal structure via the self-assembling lamination of very small objects on the two-dimensional photonic crystal structure, the occurrence of photoelectric current with semiconductor particle and the like are allowed at the immobilized states thereof on the carrier.

In a second aspect of the invention, a material for optical immobilization in the first aspect is a material containing a dye structure with azo group.

The type of the material with the capability of photoinduced deformation includes but is not limited to a material containing a dye structure with azo group, particularly preferably. The dye structure with azo group is exposed to cis-trans isomerization under light or the like, so that the movement at the molecular level due to the isomerization plasticizes the material for optical immobilization, leading to ready deformation. Such action occurs particularly greatly in a dye structure having an azobenzene backbone.

In a third aspect of the invention, the dye structure with azo group in the second aspect is an azobenzene structure having an aromatic ring containing one or more electron donating substituents with negative substituent constants according to the Hammet's rule and an aromatic group containing one or more electron withdrawing substituents with positive substituent constants according to the Hammet's rule, which are indivisually on both sides of the azo group.

The Hammet's rule expressed by the formula “log (K/K₀)=ρσ” has been known. In the formula, K relates to the reaction of m-, p-substituted phenyl compounds (K is for example the ionization constant of non-substituted benzoic acid). ρ is the proportion constant relatively representing the ratio of the electron withdrawing property:the electron donating property, as required for a certain reaction. When the substituent constant σ is positive, it means the substituent is an electron withdrawing substituent. When the substituent constant σ is negative, it means the substituent is an electron donating substituent. The value of the substituent constant σ slightly varies in references. One example thereof is shown below in Table 1. This Table 1 is cited from J. Hine, “Physical Organic Chemistry”, McGraw-Hill (1956), p.72.

TABLE 1

| Substituent | σ |
|---|---|
| p-O⁻ | −1.000 |
| m-O⁻ | −0.710 |
| p-NH₂ | −0.860 |
| p-(CH₃)N | −0.600 |
| p-OH | −0.357 |
| p-CH₃O | −0.288 |
| m-(CH₃)₂N | −0.211 |
| p-(CH₃)₃C | −0.197 |
| p-CH₃ | −0.17 |
| m-NH₂ | −0.161 |
| p-C₂H₅ | −0.151 |
| p-(CH₃)₂CH | −0.151 |
| m-(CH₃)₂Si | −0.121 |
| m-(CH₃)₃C | −0.120 |
| p-(CH₃)₂Si | −0.072 |
| m-OH | −0.069 |
| p-CH₃S | −0.047 |
| p-C₆H₅O | −0.028 |
| p-NHCOCH₃ | −0.015 |
| m-OH | −0.002 |
| p-C₆H₅ | 0.009 |
| p-F | 0.082 |
| m-CO₂ | 0.100 |
| m-CH₃O | 0.115 |
| p-CO₂ | 0.130 |
| m-CH₃S | 0.144 |
| m-C₆H₅ | 0.218 |
| p-Cl | 0.226 |
| p-Br | 0.232 |
| p-I | 0.276 |
| m-F | 0.337 |
| m-COO₂H | 0.355 |
| m-CF₃ | 0.415 |
| p-CH₃CO | 0.516 |
| p-COO₂Et | 0.522 |
| m-CH₃SO₂ | 0.647 |
| m-CN | 0.678 |
| p-CN | 0.628 |
| m-CH₃CO | 0.706 |
| m-NO₂ | 0.710 |
| P-NO₂ | 0.778 |

When the dye structure with azo group has an electron withdrawing group and an electron donating group as in the third aspect, the cis-trans photoisomerization more readily occurs, leading to more ready occurrence of photoinduced deformation due to the plasticization of the material for optical immobilization. When an electron withdrawing functional group is attached to one benzene ring in the azobenzene backbone and an electron donating functional group is attached to the other benzene ring therein, the resulting dye structure repeats isomerization between the trans form and the cis form during optical irradiation (photoisomerization cycle). Thus, the plasticization of the material for optical isomerization is then more distinct.

At the plasticized state of the material for optical immobilization, an electromagnetic field based on irradiation light or an electromagnetic field from electrodes interacts with the material for optical immobilization at the plasticized state thereof; or an electromagnetic field formed around the very small object or an electrostatic force, van der Waals force or atomic force based on the presence of the very small object interacts with the material for optical immobilization at the plasticized state thereof. Consequently, the material for optical immobilization deforms optically.

In a fourth aspect of the invention, the dye structure with azo group in the third aspect is a dye structure under control so that the cut-off wavelength of the photoabsorption wavelength on the side of longer wavelength may exist in the region of shorter wavelength than the fluorescence peak wavelength of a fluorescence dye for fluorescence analysis, provided that the dye structure has the electron withdrawing substituents and the electron donating substituents under conditions that the following formula 1 can be established.

Σ|σ|≦|σ1|+|σ2| [Formula 1]

In the formula 1, σ is the substituent constant according to the Hammet's rule; σ1 is the substituent constant of cyano group; and σ2 is the substituent constant of amino group.

As in the fourth aspect, the material for optical immobilization can be a material containing a dye structure with modifications of the intrinsic absorption wavelength. Generally, the absorption wavelength of the dye structure with aromatic rings such as azobenzene shifts to the longer wavelength, when the dye structure has predetermined electron withdrawing functional groups and electron donating functional groups. When the electron withdrawing properties of these groups and the electron donating properties thereof are stronger, the shift ratio is larger.

As the sum of the absolute values of all the substituents σ used for substitution in the dye structure is larger, generally, the absorption wavelength shifts to the longer wavelength. By selecting such substituents so that the value Σ|σ| in the formula 1 may be a given value or less, a dye structure with the cut-off wavelength of absorption wavelength on the side of longer wavelength as controlled to be in the region of shorter wavelength than the fluorescence peak wavelength of fluorescent molecule for fluorescent analysis can be obtained. In case of a combination of p-nitro group and p-amino group (tertiary), the value Σ|σ| in the formula 1 is 1.378 according to Table 1, while the cut-off wavelength then is 650 nm. In a case of p-cyano group and p-amino group (tertiary), on the other hand, the value Σ|σ| is 1.228, while the cut-off wavelength then is 570 nm. By selecting a combination of substituents so that the value Σ|σ| is 1.228 or less on the basis of the value σ of each of the substituents, a dye structure with the cut-off wavelength of 570 nm or less can be designed.

According to the fourth aspect, the absorption band causing photoinduced deformation of the material for optical immobilization never overlaps with the fluorescence band of a fluorescence dye for fluorescence analysis. Therefore, any disadvantage of no detection of fluorescence due to the fluorescence absorption via the dye structure of the material for optical immobilization can be avoided during the fluorescence analysis using a fluorescence dye after the optical immobilization of very small objects.

More specifically, indodicarbocyanide series Cy3 and Cy5 as practically effective fluorescence dyes or various cyanine dimer-series fluorescence dyes or various cyanine monomer-series fluorescence dyes manufactured by Molecular Probe Co. or a series of Alexa Fluoro fluorescence colors have fluorescence peaks at wavelength of 565 nm or more. In this case, therefore, the actions and advantages described above can be secured when the cut-off wavelength of the photoabsorption of the dye structure in the material for optical immobilization on the side of longer wavelength is 570 nm or less.

The carrier having optically immobilized very small objects by the method in the fourth aspect can be used for example as a fluorescence sensor as a sensor of importance in the field of biochemistry. More specifically, the carrier can be used as evanescent-wave sensor of waveguide type for detecting antigen-antibody reaction or as DNA microarray or DNA chip for the multivariate analysis of gene function or the like, as follows. In other words, a substance (ligand) specifically binding to a test subject substance (analyte) is immobilized as a very small object on the carrier. The analyte having been preliminarily bound with a fluorescence substance by an appropriate approach is then bound to the ligand, to detect the fluorescence.

The method for immobilizing ligands on carriers so far includes a process of forming chemical bonding (covalent bonding), a process using physical adsorption (hydrophobic interactions and electrostatic interactions) and the like. However, the former process requires the use of activating reagents for the formation of such bonding, involving complicated procedures or causing concerns about the denaturation or inactivation of the ligand under some reaction conditions for the formation of the bonding, when the ligand is a biomolecule. The latter process potentially causes the detachment of the ligand because the adsorptivity is insufficient. The method in the fourth aspect can avoid such disadvantages.

In a fifth aspect of the invention, the very small object in the first to fourth aspects of the invention is one or two or more selected from the following groups (1) to (5).

(1) An inorganic material particle group. The group includes at least metal particles, metal oxide particles, semiconductor particles and ceramics particles.

(2) An organic material particle group. The group includes at least plastic particles.

(3) A high-molecular organic molecule group. The group includes at least polypeptide molecules in chain, protein molecules with active-type or inactive-type configurations, assemblies of these protein molecules, single-stranded or double-stranded or higher-stranded nucleic acid molecules, or polysaccharide molecules.

(4) Fine particles of inorganic materials or organic materials, to which high-molecular organic molecules are preliminarily bound.

(5) Cells, organellas, bacteria, viruses, biological tissues or biological organisms. At least the cells, bacteria or biological organisms in the group include those at viable state.

One group of the typical preferable example of the very small object is the inorganic material particle (1) in the fifth aspect. The group includes at least metal particles, metal oxide particles, semiconductor particles and ceramic particles. An additional group of the typical preferable example of the very small object is the organic material particle (2) in the fifth aspect. The group includes at least plastic particles. These particles can be immobilized in a simple manner without any use of binders such as gel substances or the like.

Another group of the typical preferable example of the very small object is the high-molecular weight organic molecule (3) in the fifth aspect. The group includes at least polypeptide molecules in chain, protein molecules with active-site or inactive-site, or single-stranded or double-stranded or higher-stranded nucleic acid molecules. Preferably, the protein as the very small object includes for example appropriate proteins, enzymes, antigens, antibodies or cell membrane receptors expressed in biological organisms. These polypeptides can be immobilized with no damage on the configurations (namely, the intrinsic activities or functions) via optical immobilization. Intentionally, a protein molecule with a configuration of inactive type can also be immobilized.

The single-stranded or double-stranded or higher-stranded nucleic acid molecules as very small objects include for example mRNA or fragments thereof, cDNA or fragments thereof, fragments of genome DNA, DNA fragments including single nucleotide polymorphism, restriction fragments or DNA fragments including microsatellite parts. For DNA chips so far, for example, these polynucleotides are spotted at their state in aqueous solution on a carrier and then dried thereon, for immobilization. However, the method requires surface treatment of the carrier, so as to enhance the DNA adherability of the carrier surface. Additionally, the method requires the blocking treatment. Therefore, DNA is readily detached from the carrier. According to the fifth aspect, polynucleotides can sufficiently be immobilized with no specific pretreatment or post-treatment of the carrier.

Like (4) in the fifth aspect, an immobilization mode of preliminarily binding a fine particle of an inorganic material or an organic material to the end of polynucleotide and then immobilizing the fine particle on the carrier is also preferable. The immobilization mode is particularly preferable in case that the polynucleotide immobilization encompasses the purpose of hybridization with homologous or complementary polynucleotides. In this case, the fine particle bound to the end of polynucleotide can be immobilized at an appropriate position on the carrier, using for example laser trapping.

Another group of the typical preferable example of the very small object includes the cells, biological tissues or biological organisms as described as (5) in the fifth aspect. At least the cells or biological organisms in the group include those at viable state. In case of using cells or microorganisms for in vitro analysis, particularly, it is required to immobilize these at viable state. Such requirement can be satisfied readily when the immobilization is done in water or aqueous buffers or the like.

In a sixth aspect of the invention, the arrangement and immobilization of very small object on the surface of a carrier in the first to fifth aspects of the invention is carried out in a liquid medium dissolving or suspending the very small object therein.

In the sixth aspect of the invention, the type of the “liquid medium” is not limited. However, generally, water or solutions of a composition with the main medium water are preferably used.

The arrangement and immobilization of very small object on the carrier surface can be done in appropriate forms. Particularly preferably, the arrangement and immobilization thereof is done in liquid media dissolving or suspending very small object. The reason is that very small object can spread readily over the surface of the carrier immersed in the liquid media, so that very small object can be immobilized in liquid media optimal for the functional retention and viability of the very small object such as protein, cell, and microorganism.

The liquid media in the sixth aspect particularly preferably include water or solutions of a composition with the main medium water. The solutions of a composition with the main medium water preferably include for example buffers, pH-adjusted buffers, solutions dissolving nutritious components for cells or microorganisms therein and the like.

In a seventh aspect of the invention, laser trapping is used for arranging very small object on the surface of a carrier in the first to sixth aspects.

Laser trapping means an approach for trapping objects at a position with a higher laser intensity portion, using radiation pressure. Converging light of a wavelength with which a carrier reacts on the surface of the carrier for irradiation, very small object is trapped on the converged portion and is then immobilized at the position on the surface of the carrier. It is also possible to capture the very small object with a beam of a wavelength with which a carrier is unreactive and then to immobilize the very small object with a beam of a wavelength with which the carrier is reactive.

In an eighth aspect of the invention, a great number of one type or two or more types of very small objects can be immobilized following specific distribution patterns differing from each other on the surface of a carrier, by giving a preset distribution to the irradiation region or irradiation intensity of irradiation light using an appropriate tool, according to the methods in the first to sixth aspects of the invention.

For the immobilization and patterning of a great number of two or more types of very small objects according to specific patterns differing from each other in the eighth aspect, a process of immobilizing a great number of each type of very small objects on the carrier and sequentially repeating the step may be satisfactory. If possible, such process may be satisfactorily done simultaneously for each type of very small objects.

As the tool for giving preset patterns of light intensity distribution, for example, photomask and/or interference light is used. For the use of photomask, there can be employed the proximity exposure method using photomask in adhesion and the projection exposure method using photomask in no adhesion, which is dominant in recent semiconductor lithography.

When preset patterns of light intensity distribution are given to the carrier surface, very small objects per se are also immobilized on the carrier surface, following the preset pattern. Thereby, very small objects can be immobilized so that the region of the immobilized very small objects may form a specific circuit or the like.

Further, using very small objects of different types, for example, the immobilization method is repetitively done according to different patterns (if possible, the method is done in a simultaneously progressing manner), so that plural types of circuits of various modes and with diverse functions and the like can be formed appropriately.

As the tool for giving preset patterns of light intensity distribution, the use of photomask by the proximity exposure method and the projection exposure method or the use of interference light is particularly preferable. Examples of the use of photomask are shown in FIG. 1A to FIG. 1D, while examples of the use of interference light are shown in FIG. 2A to FIG. 2D.

In FIG. 1A, liquid medium 2a containing very small objects is placed on carrier 1. After coating photomask 3a with a specific light transmission pattern over the liquid medium 2a, the photomask is irradiated with irradiation light 4. As shown in FIG. 1B, consequently, very small objects 5a are immobilized on the carrier 1, following the preset light transmission pattern. As shown in FIG. 1C, then, liquid medium 2b containing a different type of very small objects 5b are arranged on the carrier 1. After coating photomask 3b with a different light transmission pattern over the liquid medium 2b, the photomask is irradiated with irradiation light 4. As shown in FIG. 1D, consequently, very small objects 5b are immobilized on the carrier 1 following the preset light transmission pattern, together with the very small objects 5a immobilized as described above. In such manner, appropriate types of very small objects can be immobilized in appropriate patterns on one carrier.

In FIG. 2A, liquid medium 7a containing very small object 6a are arranged on carrier 1. The liquid medium 7a is irradiated with interference light 8 (for example, interference light of two-beam interference) as irradiation light. As shown in FIG. 2B, consequently, very small objects 6a are immobilized on a carrier 1 according to intensity distribution of the interference light. As shown in FIG. 2C, then, liquid medium 7b containing a different type of very small object 6b are arranged on the carrier 1. Then, the liquid medium 7b is irradiated with full irradiation light 9. As shown in FIG. 2D, consequently, the very small object 6b is immobilized on the whole surface of the carrier 1, together with the very small object 6a immobilized as described above. In FIG. 2C, interference light with a distribution pattern differing from that of the interference light 8 and a photomask with an appropriate light transmission pattern formed thereon may be used in place of the full irradiation light 9.

In a ninth aspect of the invention, the irradiation light in the first to eighth aspects is propagating light, optical near field or evanescent field.

Essentially, the type of the irradiation light is not limited. Various types of propagating light, optical near field or evanescent field can be used appropriately. However, in some case, the wavelength or intensity or the like of irradiation light is limited, so as to cope with the type of the material type with the capability of photoinduced deformation. Additionally, the use of propagating light is limited in some case in terms of the relation with the size of very small object.

As well known, propagating light cannot be converged to a size of about the wavelength of light or less, even by using any lens, because propagating light has the property of wave. Thus, a very small object can never be immobilized at a precision position within the diffraction limit of light. Optical near field has not any such limitation. Very small object can thereby be immobilized in a micro-scale region of the nanometer order within the diffraction limit of light. When an optical fiber probe for optical near field microscopy is used as a source of optical near field, very small object can be immobilized in a region of 50 nm or less at an appropriate position on the carrier.

Evanescent field means an electromagnetic field penetrating at a distance of about the wavelength of light along the adverse direction of reflection light during total reflection of light. Examples of the use of evanescent light are shown in FIG. 3. Liquid medium 2 containing very small objects 5 is placed on carrier 1. On the underside of the carrier 1 is placed prism 12. Injection light 10 irradiating the prism 12 along the direction shown in the figure is reflected on the underside of the carrier 1, to become reflection light 11. Then, evanescent light penetrates through the topside face of the carrier 1, so that very small objects 5 are immobilized on the carrier 1.

In a tenth aspect of the invention, a carrier having immobilized very small objects thereon is provided, where very small object is immobilized on the surface of the carrier by a method for optically immobilizing very small object in accordance with any of the first to the ninth aspects of the invention.

A very small object-immobilized carrier where very small object has been immobilized on the surface of the carrier by a method for optically immobilizing very small object in accordance with the first to the ninth aspects of the invention, can be provided readily at low cost. Furthermore, a carrier having immobilized thereon diverse types of very small objects involving so far difficulty in their immobilization, according to diverse immobilization patterns, can be provided. Furthermore, specific functions and viable states of very small objects having been immobilized can be maintained.

In an eleventh aspect of the invention, further, the carrier having immobilized very small object in accordance with the tenth aspect is an integrated circuit chip, where any very small object (1) or (2) in the fifth aspect is immobilized on an integrated circuit substrate as the carrier, following a preset distribution pattern.

One of the preferable typical examples of the carrier having immobilized very small objects thereon is an integrated circuit chip immobilizing metal particles, metal oxide particles, semiconductor particles, silica particles or plastic particles on an integrated circuit substrate as the carrier, following a preset distribution pattern.

In a twelfth aspect of the invention, the very small object-immobilized carrier in accordance with the tenth aspect is the following (6) or (7).

(6) A bioreactor or biosensor prepared by immobilizing very small object, namely a single species or plural species of enzymes, antibodies, antigens, microorganisms, or organellas on the carrier as a reaction bed or a substrate.

(7) A bioassay test piece or a protein chip for proteome analysis, as prepared by immobilizing protein expressed in biological cells. Such protein includes for example antigen, antibody, cell membrane receptor or various functional proteins expressed in tissue-specific, diseased condition-specific or development/differentiation stage-specific manners in biological organisms. Further, the term “proteome analysis” is a concept including the structural analysis of proteins and the analysis of protein interactions.

Other preferable typical examples of the carrier having immobilized thereon very small objects include carriers having immobilized various types of proteins. Particularly preferable examples are the bioreactor or biosensor (6) and the bioassay test piece or protein chip for proteome analysis as (7) in the twelfth aspect.

The functions of various types of proteins are frequently based on the specific delicate configurations thereof. The functions are readily deteriorated, for example via the chemical treatment for general immobilization and external stimulation with pH, heat and the like. However, the optical immobilization of the invention causes such concerns less. On the other hand, various types of proteins have molecular surfaces individually differing in terms of physical and chemical properties. Therefore, general immobilization requires carriers with surface profiles coping with various types of proteins. The optical immobilization in accordance with the invention essentially never depends on the properties of the surface of protein molecule.

In a thirteenth aspect of the invention, the bioreactor or biosensor (6) in the twelfth aspect is a carrier having immobilized very small object thereon, where the very small object (6) is immobilized on the surface of the carrier using the material for optical immobilization at least on the surface layer thereof and where electrodes are formed on the surface of the carrier.

Reactors or sensors using electrochemical reactions have been studied traditionally. In recent years, attention has been focused on bioreactor or biosensor electrochemically transforming the selective reaction of a biological substance typically including enzyme into electric signal. For the bioreactor or biosensor, it is one of important techniques to immobilize a biological substance for use in the reaction in the proximity of the electrode. Further, the biological substance is never inactivated then. In the course of developing the use of bioreactor or biosensor, still additionally, downsizing, multi-functional preparation, and integration are very significant issues therefor.

In bio-electrochemical bioreactors or biosensors so far, biological substances are immobilized using for example specific spacers or are immobilized on an oxide film on silicon substrate or a conductive polymer or the like. In any of the cases, however, the immobilization of biological substance via chemical bonding is essential, leading to potential inactivation of biological substance. Additionally, a drawback exists that the production process is generally complicated. The very small object-immobilized carrier in the thirteenth aspect can avoid such problems.

In a fourteenth aspect of the invention, the carrier in the bioassay test piece or protein chip for proteome analysis as (7) in the twelfth aspect is accompanied with a film of the material for optical immobilization, which is formed on the surface of a metal thin film with the occurrence of surface plasmon resonance phenomenon and is a very small object-immobilized carrier having immobilized the protein as the very small object on the surface of the carrier.

SPR sensor based on the SPR (surface plasmon resonance) method currently exists as one of important sensors in the field of biochemistry. According to the method, the SPR phenomenon is used, such that when light is reflected off a thin metal film (for example, a film thickness of 100 nm or less) under total reflection conditions, the metal film resonates with light at a certain specific angle, generating a surface plasmon wave. The angle where the SPR is observed is extremely sensitive to the refractive index in the periphery of the metal. The energy of incident light is consumed for the excitation of SPR, so that the intensity of the reflection light is reduced. When functional proteins (ligands) as very small objects immobilized on the surface of a carrier specifically binds to a test subject material, the refractive index changes. Thus, the change of the refractive index can be detected in a sensitive manner.

The metal film may be of a single layer structure or of a laminate structure of two layers or more. The film thickness is any appropriate thickness but is preferably 200 nm or less, particularly preferably 100 nm or less. On the surface of the metal film on the opposite side of the face where the film of the material for optical immobilization is formed, a transparent medium layer of glass and the like is preferably arranged.

In a fifteenth aspect of the invention, the very small object-immobilized carrier in the tenth aspect is any of the following carriers (8) to (10).

(8) A DNA chip or DNA microarray immobilizing DNA fragments usable as a genetic marker thereon.

(9) A DNA chip or DNA microarray immobilizing DNA fragments including DNA fragments containing single nucleotide polymorphism (SNP), restriction fragments or DNA fragments containing microsatellite part thereon.

(10) A DNA chip or DNA microarray immobilizing mRNA or fragments thereof, cDNA or fragments thereof, or fragments of genome DNA thereon.

One of other preferable typical examples of the very small object-immobilized carrier is a carrier immobilizing various types of polynucletides thereon. A particularly preferable example is each type of DNA chips or DNA microarrays as (8) to (10) in the fifteenth aspect.

In a sixteenth aspect of the invention, a method for observing very small objects including a step of immobilizing very small objects on the surface of a carrier by a method for optically immobilizing very small objects in the first to ninth aspects, and a step of observing the very small objects immobilized by an appropriate process of giving displacement force to the very small object.

In the sixteenth aspect, the “displacement force” means an appropriate type of force, which exerts an action to displace very small object from the arranged or immobilized position to a different position. The displacement force includes for example the physical contact of other materials to very small object, atomic force, electric force, magnetic force, abrasion force and optical radiation pressure interactive with very small objects.

Due to the higher immobilization force, very small objects immobilized on the surface of a carrier by the individual methods for optically immobilizing very small objects hardly displace, under observation by an appropriate method for giving the very small objects the displacement force (for example, scanning probe microscopy). Consequently, the very small objects can be observed at high precision in a reliable manner.

In a seventeenth aspect of the invention, a method for observing very small objects is provided, which enables the observation of very small objects at viable state while it is immobilized as it is, when the very small object in the sixteenth aspect is a cell or a microorganism.

As described above, the immobilization of very small object in case that the very small objects are cells or microorganisms is done for example in a buffer, which enables the observation thereof at viable state. In case of the in vitro analysis of the function of a functional biomolecule using a cell or a microorganism, this is very significant.

In an eighteenth aspect of the invention, a method for observing very small objects in case that the very small objects in the sixteenth aspect are enzymes, antigens, antibodies or cell membrane receptors as polypeptide is provided, including the use of a scanning probe microscope as an observation tool to modify the probe with an enzyme substrate, an antibody, an antigen or a cell membrane receptor ligand to thereby analyze the reactive part of the enzyme, the antigen, the antibody or the cell membrane receptor, functionally or in terms of steric configuration.

The observation method in the eighteenth aspect enables the analysis of the activity center of the enzyme in terms of configuration because a predetermined change is induced in the substrate just when the probe for example reaches the activity center of the enzyme. Otherwise, the observation method in the eighteenth aspect enables the determination of the substrate specificity of the enzyme. Even in case of antigen, antibody or cell membrane receptor, the same actions and advantages can be expected.

The above and other advantages of the invention will become more apparent in the following description and the accompanying drawings.

### BRIEF DESCRIPTION OF THE DRAWINGS

FIG. 1A to FIG. 1D depict the modes for carrying out the invention. FIG. 2A to FIG. 2D depict the modes for carrying out the invention. FIG. 3 depicts the mode for carrying out the invention. FIG. 4 depicts an atomic force microscopic image in an example of the invention. FIG. 5 depicts an atomic force microscopic image in an example of the invention. FIG. 6 depicts an atomic force microscopic image in an example of the invention. FIG. 7 is an atomic force microscopic image enlarging the important part of FIG. 6. FIG. 8 is an atomic force microscopic image enlarging the important part of FIG. 7. FIG. 9 depicts an atomic force microscopic image in an example of the invention. FIG. 10 depicts an atomic force microscopic image of a comparative example. FIG. 11 depicts an atomic force microscopic image of an example of the invention. FIG. 12 depicts an atomic force microscopic image of an example of the invention. FIG. 13 depicts an atomic force microscopic image of a comparative example. FIG. 14 depicts an atomic force microscopic image of a comparative example. FIG. 15 depicts a dark field microscopic image of an example of the invention. FIG. 16 depicts the spectral chart of an example of the invention. FIG. 17 depicts the spotting design of the DNA microarray of an example of the invention. FIG. 18A and FIG. 18B show the results of fluorescence observation in an example of the invention. FIG. 19A and FIG. 19B show the results of fluorescence observation in an example of the invention. FIG. 20 depicts the biosensor carrier for the SPR test method in an example of the invention. FIG. 21 depicts the antigen spotting design of an example of the invention. FIG. 22 depicts the result of fluorescence observation in an example of the invention. FIG. 23 shows the result of fluorescence observation in an example of the invention. FIG. 24 shows the result of fluorescence observation in an example of the invention. FIG. 25 shows the result of fluorescence observation in an example of the invention.

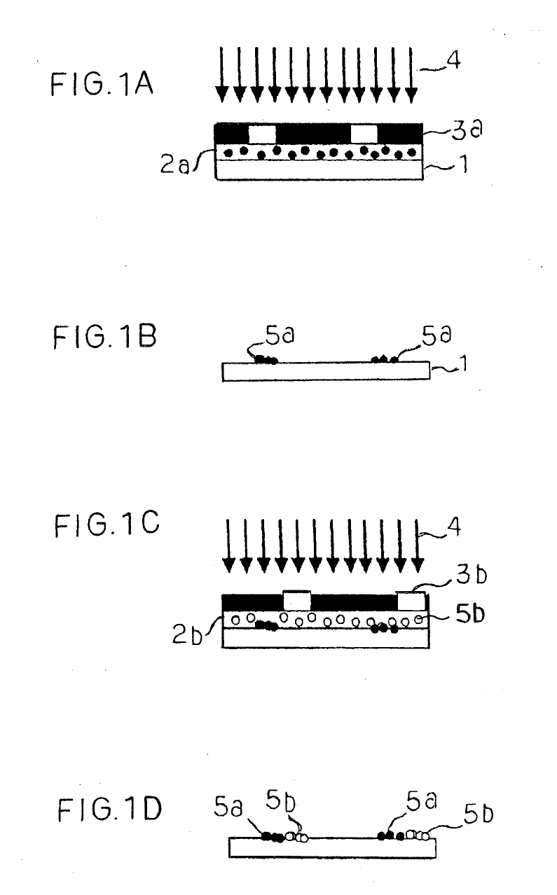

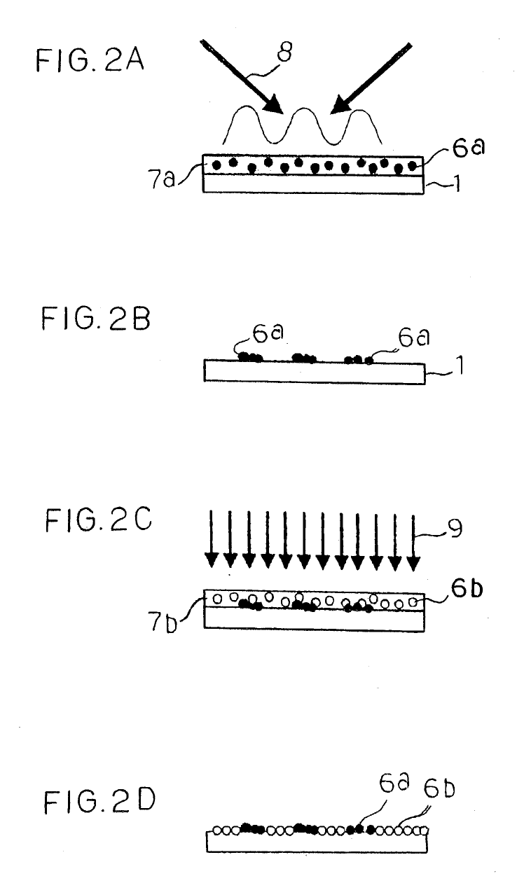

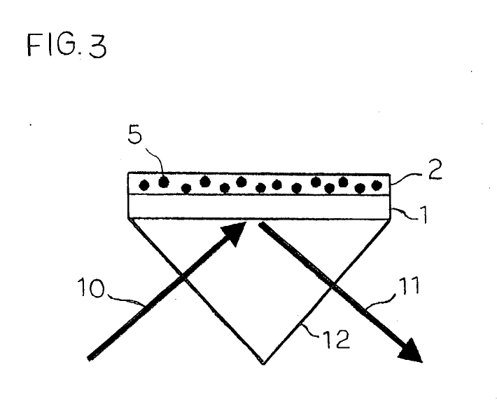

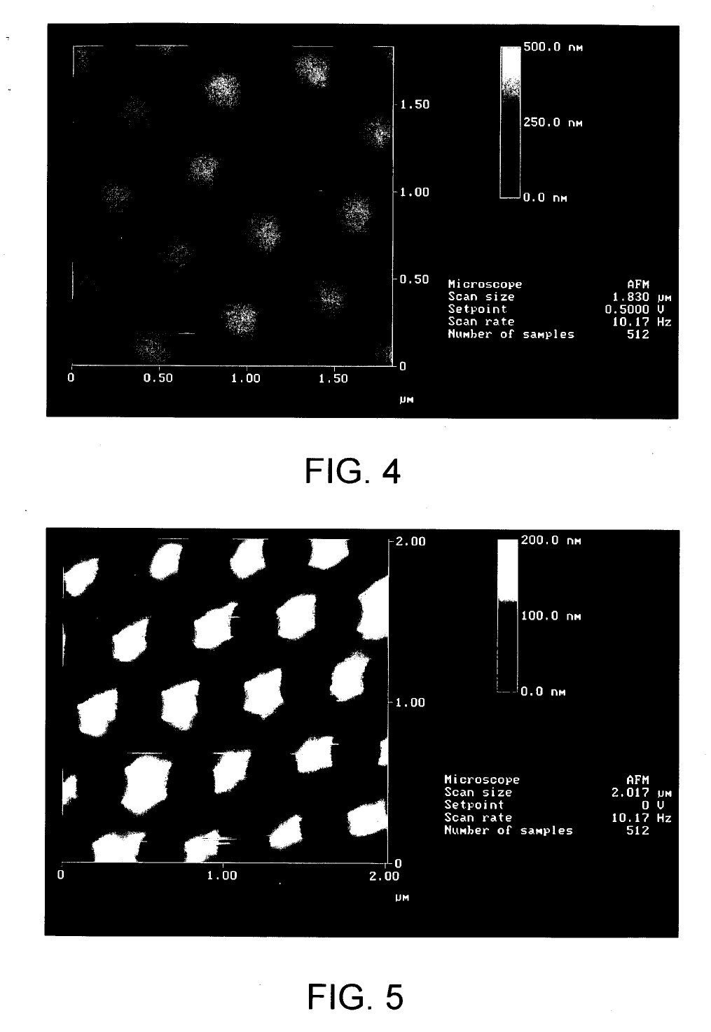

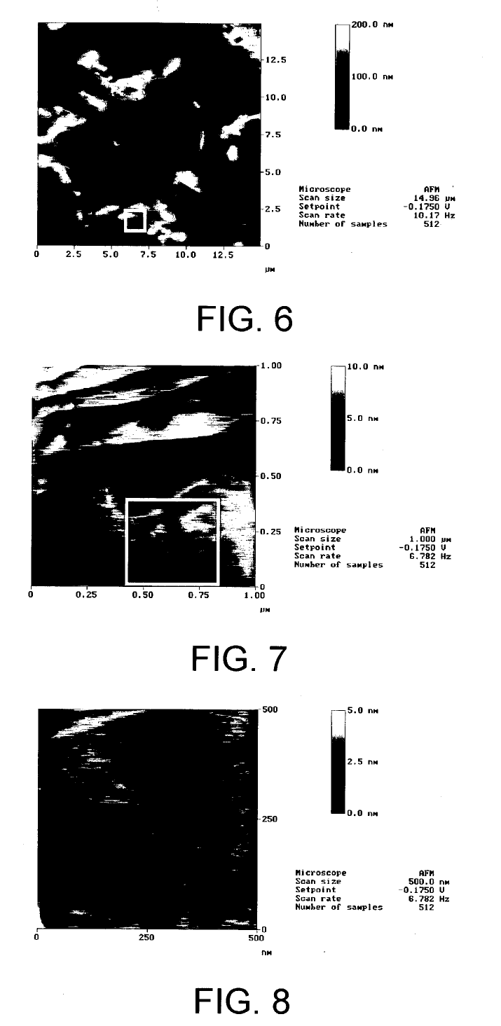

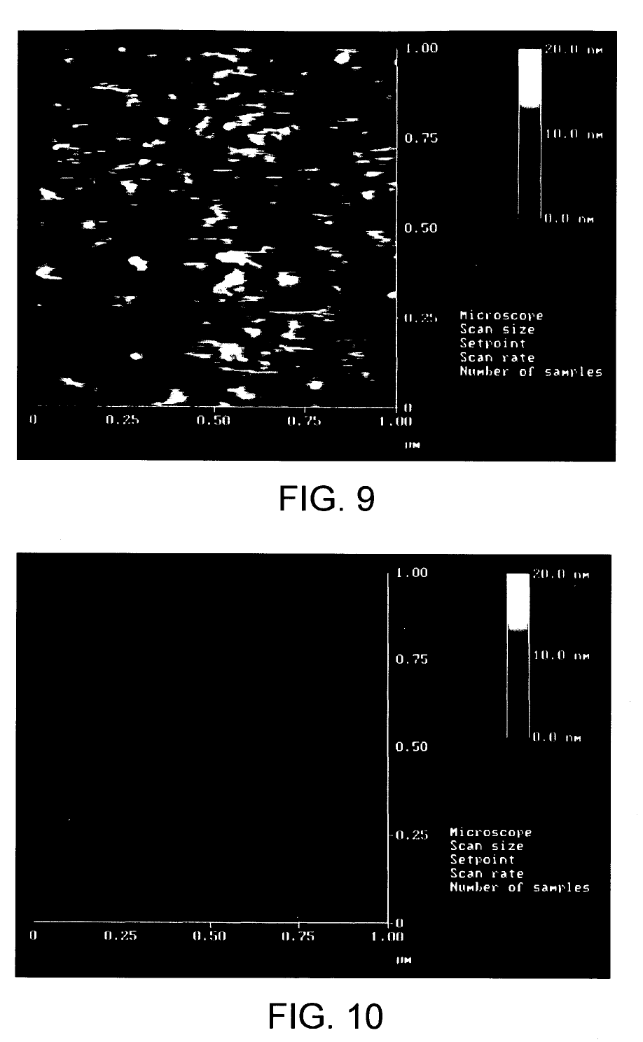

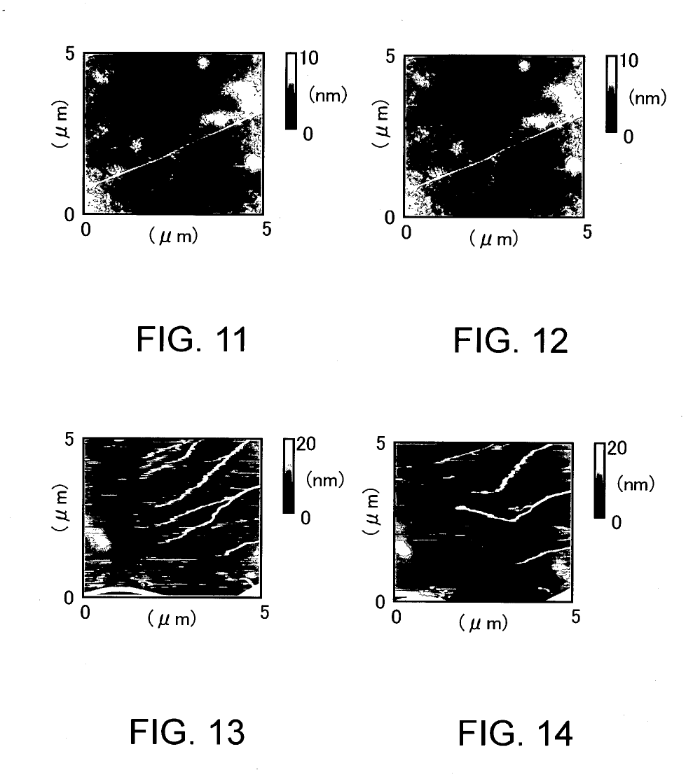

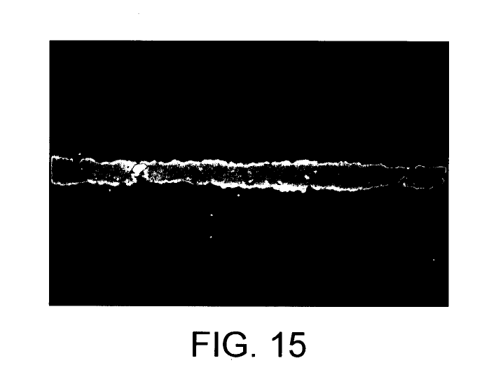

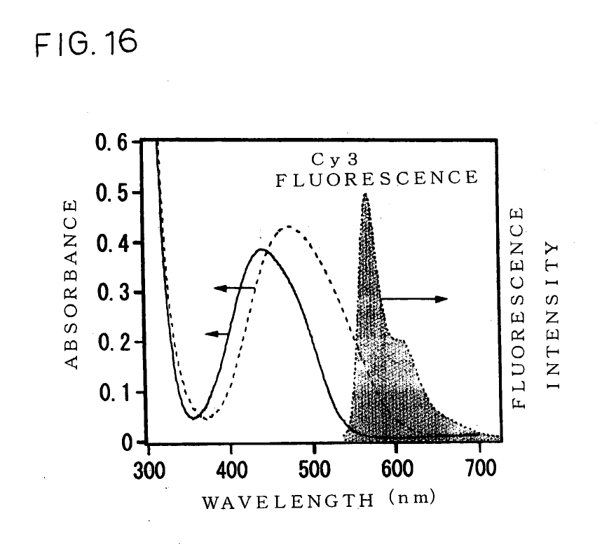

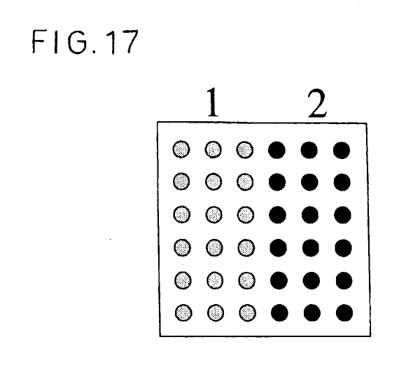

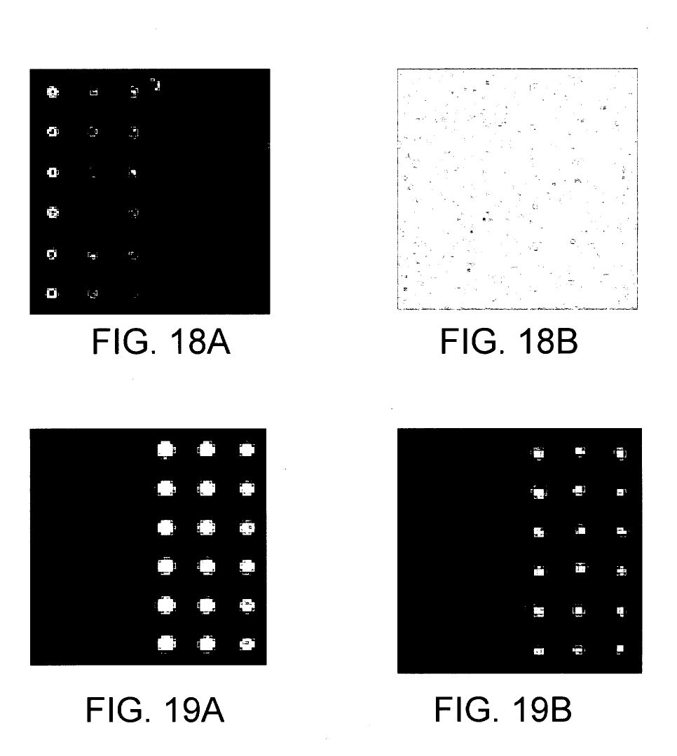

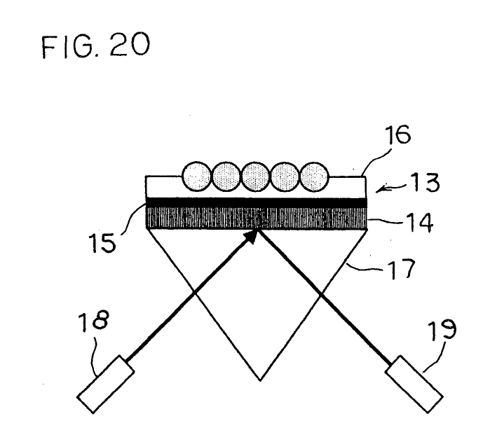

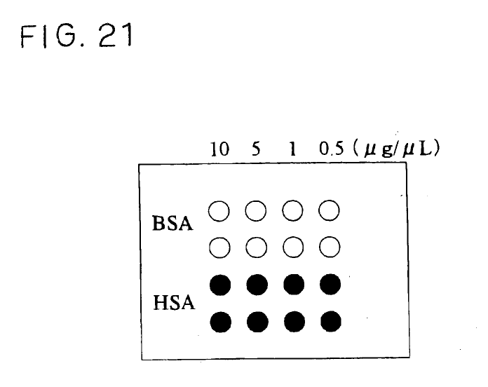

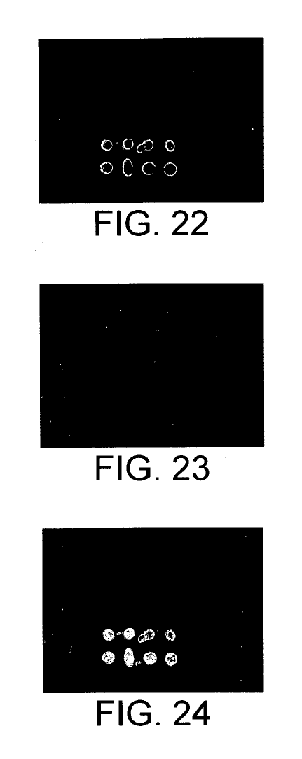

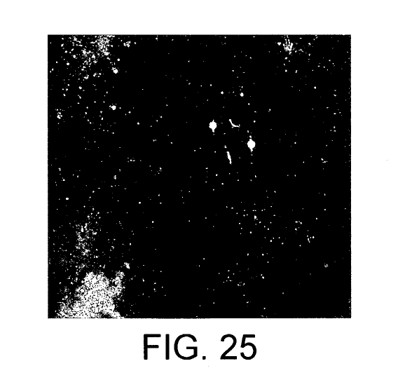

### DETAILED DESCRIPTION OF THE INVENTION

[Method for Optically Immobilizing Very Small Object]

The method for optically immobilizing very small objects in accordance with the invention uses a carrier using a material with the capability of photoinduced deformation, namely a material for optical immobilization which can exert the potency for immobilizing very small objects arranged on the surface during light irradiation, at least on the surface layer thereof. While the very small object as a shaped material of a size of 50 μm or less (more preferably 10 μm or less, still more preferably 1 nm or more) is arranged on (put in contact to) the surface of the carrier, the very small object is immobilized on the surface of the carrier via irradiation with light.

Any mode for arranging very small objects on the surface of the carrier is satisfactory. For example, very small objects may be at just sprinkled states on the surface of the carrier or may be at electrostatically adhering states or the like. Particularly preferably, very small objects in liquid media such as water are arranged on the surface of a carrier. In case of desiring good spread of a great number of very small objects on the surface of a carrier or in case that the intrinsic functions of very small objects may potentially be deteriorated (for example, protein denaturation, the death of cells and the like) when the very small objects are dried, particularly preferably, the arrangement of very small objects is done in liquid media. The arrangement is done for example in a mode of immersing a carrier in a liquid medium dissolving or suspending very small objects therein or a mode of dropwise adding a small volume of the liquid medium to the surface of a carrier. When very small objects are proteins or viable cells, particularly preferably, the liquid medium is a buffer with appropriate pH, ionic strength, nutrient composition and the like.

Individuals of very small objects may be immobilized separately. A great number of very small objects may be immobilized following a specific distribution pattern. Very small objects may sometimes be arranged at their self-assembling state on the surface of a carrier to be then immobilized, in case that the very small objects have a self-assembly property.

[Carrier]

The carrier is made of a material for optical immobilization or is produced by using a material for optical immobilization at least as the surface layer. The form, material and use of the carrier are not limited. Some examples of the carrier form are the form of relatively small chips such as integrated circuit substrate and DNA chip; the form of particles packed in column or the like; the form of large fixative reaction bed fluidizing substrate solution and the like; forms such as the form of small-size test paper for use in simple immunoassay; and forms such as the form of slide glass for use in microscopic observation.

[Material for Optical Immobilization]

The type of the material for optical immobilization, which composes at least the surface layer of the carrier, is not specifically limited, as long as the material for optical immobilization has a capability of photoinduced deformation and can exert the potency of immobilizing the very small objects arranged on the surface during light irradiation.

Preferable examples of the material for optical immobilization include organic or inorganic materials containing components involving ablation, photochromism or photoinduced orientation of molecules (a photoreactive component) via irradiation with light. As consequence of irradiation, the volume, density or free volume of such material for optical immobilization vary, so that photoinduced deformation occurs. Additionally, inorganic materials such as generically called chalcogenite glass containing a composition of any of sulfur, selenium and tellurium as bound to any of germanium, arsenic and antimony are also exemplified.

The type of the photoreactive component preferably includes for example but is not limited to photoisomerization-potential components and photopolymerizable components as components possibly causing anisotrophic photochemical reaction involving the change of the form of the material. In case that very small object to be immobilized is a biological substance, of which the activity may be deteriorated via the chemical reaction with the carrier material, photoisomerization-potential components are preferable as the photoreactive components. Preferably, the photoisomerization-potential components include for example components causing trans-cis photoisomerization, particularly representatively dye structures with azo group (—N═N—), specifically a component with the chemical structure of azobenzene or a derivative thereof.

In case that the material for optical immobilization is a material containing dye structures with azo group, particularly preferably, the dye structure has one or two or more electron withdrawing functional groups (electron withdrawing substituents) and/or one or two or more electron donating functional groups (electron donating substituents). Most preferably, the dye structure has both these electron withdrawing functional groups and electron donating functional groups. The electron withdrawing functional groups preferably include for example such functional groups with positive values of the substituent constant σ according to the Hammet's rule. The electron donating functional groups preferably include for example such functional groups with negative values of the substituent constant σ according to the Hammet's rule.

Particularly preferably, the material for optical immobilization has the electron withdrawing substituents and the electron donating substituents under conditions where the following formula 1 can be established. This enables the provision of a dye structure controlled so that the cut-off wavelength of the photoabsorption wavelength on the side of longer wavelength may reside in the region of shorter wavelength than the fluorescence peak wavelength of a fluorescence dye for fluorescence analysis. The type of the dye structure in this case is not limited but preferably includes for example the dye structures with azo group, particularly the chemical structures of azobenzene and derivatives thereof.

Σ|σ|≦|σ1|+|σ2| Formula 1

(In the formula 1, σ is the substituent constant; σ1 is the substituent constant of cyano group; and σ2 is the substituent constant of amino group, according to the Hammet's rule.)

In the matrix of the material for optical immobilization, satisfactorily, the photoreactive component may be simply dispersed or may make a chemical bond with the constituent molecules of the matrix and the like. From the standpoint that the distribution density of the photoreactive component in the matrix can be almost completely controlled and from the standpoint of the thermal resistance of the material or the stability thereof over time, particularly preferably, the photoreactive component is chemically bonded to the constituent molecules of the matrix. As the matrix material, organic materials such as general polymer materials and inorganic materials such as glass can be used. Taking account of the uniform dispersibility or bindability of the photoreactive component in the matrix, organic materials, particularly polymer materials are preferable.

The types of the polymer materials are not limited. In terms of thermal resistance, preferably, polymer materials containing urethane group, urea group or amide group in the repeat unit of the polymer, particularly polymer materials with ring structures like phenylene group in the main chain of the polymer are preferable. The polymer material has any molecular weight or polymerization degree, satisfactorily, as long as the polymer material can be molded into a required shape. The polymerization form is any of appropriate forms, such as linear-chain polymer, branched polymer, ladder polymer, star polymer and the like. Additionally, the polymer material is any homopolymer or any copolymer. For the stability of the photoinduced deformation over time (for the maintenance of the immobilization force of very small object over time), a polymer material with a higher glass transition temperature of for example 100° C. or more is preferable. However, a polymer material with a glass transition temperature of about ambient temperature or less may also be used.

From the respects of those described above, particularly preferable several specific examples as the polymer materials containing the photoreactive component include those described in the Examples and additionally those shown by the following formulas 1 to 4. In these examples, individually, —X represents nitro group, cyano group, trifluoromethyl group, aldehyde group or carboxyl group; —Y represents —N═N—, —CH═N—, or —CH═CH—. —R— represents phenylene group, oligomethylene group, polymethylene group or cyclohexane group.

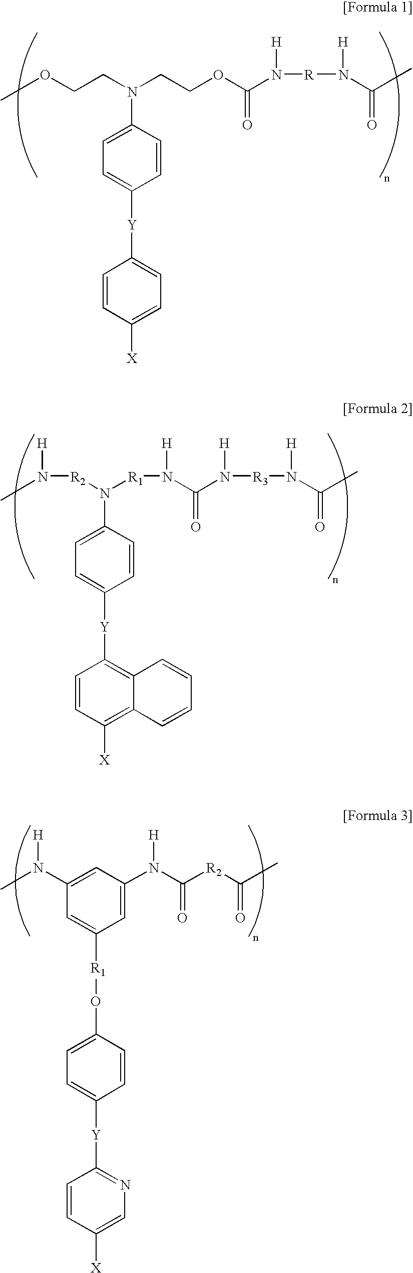

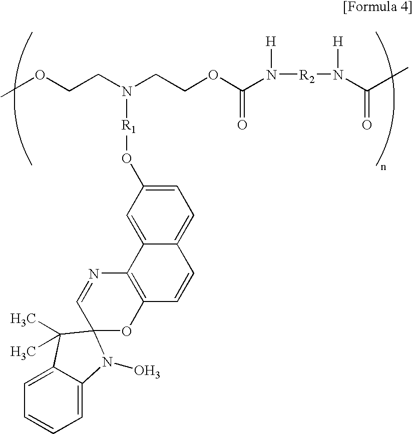

[Very Small Object]

A very small object can be a subject of a size of 50 μm or less, particularly preferably a size of 10 μm or less, specifically preferably a size of 1 nm or more. Unless a very small object is fluid (as long as a very small object has a shape), rigid materials such as metal particles to very soft materials such as animal cells can be subjects for immobilization, with no limitation.

The type of very small object preferable as the subject for immobilization is not limited but preferably includes at least one very small object selected from 1) to 4) below.

1) Metal particles, metal oxide particles, semiconductor particles, ceramic particles, plastic particles or particles of materials composed of two or more thereof (for example, a mixture of two materials or a laminate structure). Via the immobilization thereof, for example, electric field effect transistor and two-dimensional photonic crystal can be fabricated. Via the immobilization thereof according to the method for controlling the distribution pattern of immobilization as described below, additionally, electrically or optically integrated circuit chip can be fabricated for example. Silica particle or plastic particle has a potency for entrapping light. Via the immobilization thereof in waveguide, laser oscillation possibly occurs. The material with the capability of photoinduced deformation as a carrier can be used as the waveguide of a wavelength, for example a wavelength of 1.3 μm, except for the wavelength inducing photoreaction. By immobilizing silica particle or plastic particle on the material with the capability of photoinduced deformation as a carrier and guiding light on the carrier, laser can be oscillated. Device for laser oscillation can be fabricated readily.

2) Polypeptide. More specifically, very small object includes for example enzyme, antigen, antibody, and cell membrane receptor protein. Otherwise, very small object includes for example groups of various protein types to be expressed in biological cells. More preferably, very small object includes a group of proteins to be expressed in biological organisms in a tissue-specific, diseased condition-specific or development/differentiation stage-specific manners. These may be immobilized in such a manner that these are once immobilized on micro beads of plastics or the like and the resulting micro beads are immobilized on the carrier. Via the immobilization of enzyme, a bioreactor or biosensor can be fabricated. By immobilizing enzyme according to the method for controlling the distribution pattern of immobilization as described below, for example, an integrated enzyme transistor can be fabricated. By immobilizing antigen or antibody, a test chip for immunoassay and the like can be fabricated. By immobilizing cell membrane receptor protein, an analysis tool of the function of the receptor and the signal transduction mechanism of the cell can be fabricated. By immobilizing a group of various protein types to be expressed in biological cells, a test chip for proteome analysis and the like can be fabricated. By immobilizing a group of proteins to be expressed in a tissue-specific, diseased condition-specific or development/differentiation stage-specific manners, an efficacious tool enabling the analysis of protein function in relation with expression specificity can be provided.

3) Nucleic acids of single strand, double strand or higher strands (including triple strand and quadruple strand), namely polynucleotides, more specifically single-stranded polynucleotide capable of hybridization. More specifically, very small object includes for example DNA fragments including single nucleotide polymorphism, restriction fragments or DNA fragments containing microsatellite part. Because these DNA fragments can be used as genetic markers, DNA chip or DNA microarray enabling the analysis of SNP or the genotype of individual persons can be fabricated via the immobilization thereof. Additionally, such very small object can make contributions to forensic identification and the practical achievement of so-called tailor-made medicine. Very small object includes for example mRNA and fragments thereof, cDNA and fragments thereof, and fragments of genome DNA. These are also effective for gene analysis. For the immobilization of single-stranded polynucleotide and so as to enable ready hybridization, the end of the polynucleotide is particularly preferably immobilized on the carrier by preliminarily binding fine particle to the end of the polynucleotide and then immobilizing the fine particle on the carrier.

4) Cells or Microorganisms. Cells or microorganisms at viable state are particularly preferable. Via the immobilization thereof, for example, a potent tool for the in vitro analysis of the functions of biomolecules such as DNA and protein using cells or microorganisms as well as for morphological research works thereof is provided.

[Irradiating Light]

As the irradiating light, appropriate irradiating light such as propagating light, optical near field or evanescent field can be used without mismatching from the standpoint of combinations with the material with the capability of photoinduced deformation. As the propagating light, for example, natural light and laser light can be used. As propagating light, optical near field or evanescent field, the polarization profile thereof can be used. The wavelength and light source of irradiating light are not limited. Concerning the wavelength, a wavelength at a higher absorption efficiency of the material with the capability of photoinduced deformation is preferable. In case that very small objects are potentially exposed to influences such as inactivation or deterioration or the like via ultraviolet ray (wavelength of 300 to 400 nm), visible light (wavelength of 400 to 600 nm) is used as the irradiating light. Additionally, a material for optical immobilization, which can optically immobilize very small objects via the irradiation with visible light, is preferably used. The duration of irradiation with the irradiating light may appropriately be set on a needed basis. Pulse light with a high peak output may also be used.

Preferably, the optical irradiation method gives a given distribution of the irradiation region or irradiation intensity. In that case, a great number of very small objects can be immobilized on the surface of a carrier, following a preset distribution pattern. Thus, such method is effective for the fabrication of electric or optical integrated circuit chip or for the fabrication of integrated enzyme transistor. By immobilizing different types of very small objects following different distribution patterns on one carrier in a repetitive manner, a functionally complicated circuit and the like can be formed.

As a tool for giving the distribution of such irradiated region or irradiated intensity of irradiated light, for example, photomask can be used. The method for using photomask is not limited but preferably includes for example the proximity exposure method and the projection exposure method. As an additional tool for giving the distribution of such irradiation region or irradiation intensity of irradiation light, for example, the irradiation of light converged into a narrow beam can be done, following a specific pattern. Further, the intensity distribution of interference light can also be used.

As a tool for arranging very small object on a specific part on the carrier surface or for arranging a great number of very small objects on the carrier surface following a preset distribution pattern, additionally, an approach for transferring very small objects to predetermined positions by known laser trapping can also be used.

[Very Small Object-Immobilized Carrier]

Very small object-immobilized carrier of the invention is prepared by immobilizing very small objects on the surface of a carrier using a material for optical immobilization at least on the surface layer by the method for optically immobilizing very small objects. The very small object-immobilized carrier includes for example the following ones.

(Carrier Having Immobilized Particles of Inorganic Materials or Organic Materials)

Integrated Circuit Chip: Integrated circuit chip prepared by immobilizing very small objects such as metal particle, metal oxide particle, semiconductor particle, ceramics particle, and plastic particle on an integrated circuit substrate as a carrier, following a preset distribution pattern.

(Carrier Having Immobilized Protein)

Bioreactor, biosensor or integrated enzyme transistor: As prepared by immobilizing a single species or plural species of enzyme as very small objects on a carrier as a reaction bed or substrate. For example, bioreactor or biosensor with enzyme as a very small object immobilized on the surface of a carrier using a material for optical immobilization at least on the surface layer thereof and with electrodes formed on the surface of the carrier is particularly preferable.

Test Piece for Bioassay: As prepared by immobilizing antigen, antibody, cell membrane receptor, or functional protein to be expressed in biological organism in a tissue-specific, diseased condition-specific or development/differentiation stage-specific manners, as very small objects. The test piece for bioassay particularly preferably includes a test piece with a carrier on which the material for optical immobilization is formed on the surface of a metal film involving surface plasmon resonance phenomenon and where the functional protein as very small object is immobilized on the surface.

(Reaction Assay Method for Biosensor and the Like)

For the very small object-immobilized carrier having immobilized various types of protein, particularly for the bioreactor, biosensor or test piece for bioassay, the method for detecting the reaction between an immobilized protein (ligand) and a test subject analyte (analyte) is not limited.

As one of preferable detection methods, an assay method using light can be used. For example, the biosensor using the SPR method is one of preferable examples.

As an additional preferable example, the change in the refractive index via the reaction between the ligand and the analyte can be detected as the phase shift of light guided into the material for optical immobilization, by guiding light into the material for optical immobilization. For the formation of the waveguide in the carrier, then, approaches disclosed in Appl. Phys. Lett., 71, 750 (1997) and the like can be used. The detection of the change in refractive index can be done as follows. Specifically, a waveguide of Mach Zehnder type having two waveguides of an identical length is prepared, using the material for optical immobilization; a ligand is immobilized on the surface of one waveguide of them; and thereafter, an analyte solution is poured over the surface of the waveguide. In such manner, the reaction between the ligand and the analyte changes the refractive index around the waveguides, so that the phase shift of output beam can be detected.

Another preferable example is a method including a step of immobilizing a fluorescence substance on either one of a ligand and an analyte so as to detect the spectral change of the fluorescence substance and the change of the fluorescence intensity thereof, as generated via the binding of the ligand and the analyte.

A still additional preferable example is a method including a step of immobilizing a ligand on the surface of a carrier using the material for optical immobilization at least as the surface layer and a step of forming electrodes on the surface of the carrier so as to detect an electrochemically active substance as the change of electric current or electric voltage using electrode reaction, on the basis of the binding between the ligand and the analyte.

(Those Having Immobilized Nucleic Acids)

DNA Chip or DNA Microarray: Those having immobilized DNA fragments usable as genetic marker, those having immobilized DNA fragments containing single nucleotide polymorphism (SNP) regions, those having immobilized restriction fragments or DNA fragments containing microsatellite part, those having immobilized mRNA or fragments thereof, cDNA or fragments thereof, or genome DNA fragments or the like. These various types of DNA chips or DNA microarrays include those prepared by preliminarily binding a fine particle at the end of a polynucleotide, immobilizing the fine particle on the carrier to immobilize the end of the polynucleotide on the carrier.

[Method for Observing Very Small Object]

It is needless to say that very small object on the carrier can be observed by an appropriate tool. When the observation tool gives displacement force to very small object, it is difficult to observe the very small object when the very small object is not sufficiently immobilized. Depending on the size of the very small object, even the radiation pressure during observation is problematic. When scanning probe microscope is used as the observation tool, in particular, the radiation pressure is seriously problematic.

Because very small object immobilized by the immobilization method of the invention has great immobilization potency, the observation method is very advantageous in observing the very small object with an observation tool giving displacement force to the very small object. Even when very small object has the potency of autonomous movement like microorganisms, such observation method is also advantageous due to the same reason. As described above, the method is very advantageous in that very small object when it is cell or microorganism can be immobilized and observed at viable state.

In case that very small object is enzyme, antigen, antibody or cell membrane receptor as polypeptide, furthermore, scanning probe microscope is used as an observation tool to modify the probe with an enzyme substrate, an antibody, an antigen or a cell membrane receptor ligand to thereby analyze the reactive part of the enzyme, the antigen, the antibody or the cell membrane receptor functionally or in terms of steric configuration. Such analysis method is first established by the immobilization method of the invention, provided that the enzyme and the like can strongly be immobilized while the enzyme and the like are retained functionally and structurally as they are.

### EMBODIMENTS

### Example 1

(Preparation of Immobilizing Carrier)

Using a polyurethane polymer compound shown by the following formula 6, which contains the photoreactive component shown by the following formula 5, a thin film of a thickness of about 1 μm was prepared. A film-like immobilizing carrier of which the film surface is used for the immobilizing region was prepared.

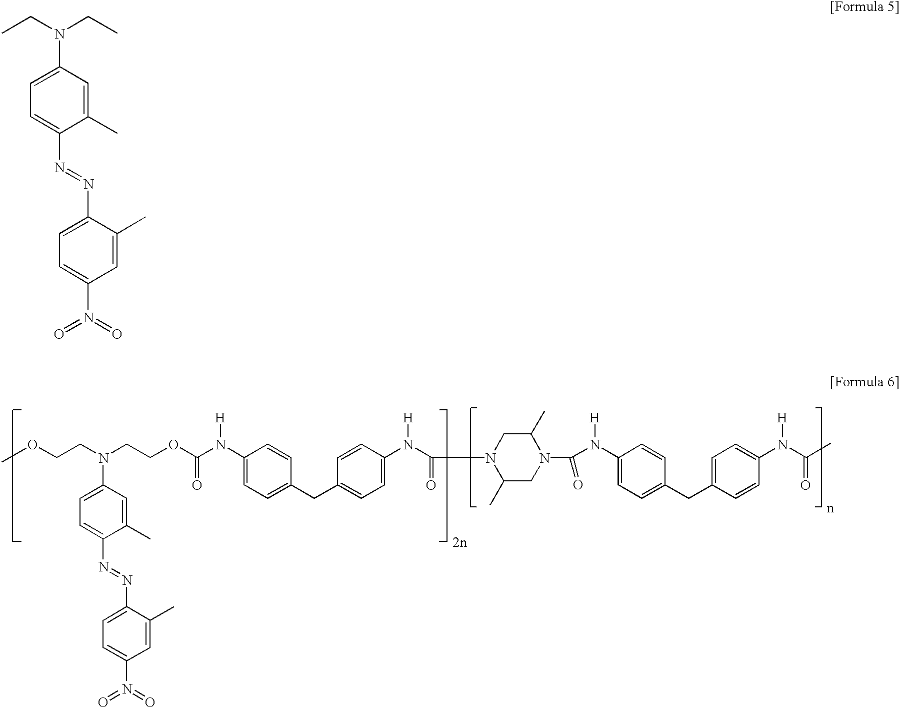

Herein, the photoreactive component shown by the formula 5 had a melting point of 169° C. The glass transition temperature of the polymer compound shown by the formula 6 was 141° C.; its intrinsic viscosity in N-methyl-2-pyrrolidone at 30° C. was 0.69 dL/g; and the wavelength at its absorption peak was 475 nm.

Additionally, the thin film was prepared by preparing a solution of the polymer compound of the formula 6 dissolved in pyridine to 6.5% by weight, filtering the resulting solution through a 0.2-μm filter, subsequently spin coating the resulting filtrate on slide glass at a rotation number of 1,000 rpm, and vacuum drying the slide glass at 80° C. for 20 hours.

(Immobilization of Polystyrene Microsphere Via Light Irradiation)

Cleaning a disc with an opened pore of a pore diameter of 5 mm by ultrasonic rinsing and subsequently mounting the disc on the immobilizing carrier, several drops of water dispersing a great number of polystyrene microspheres with diameters of 500 nm therein were added into the pore. After leaving the water to be spontaneously dried, the area where the polystyrene microsphere was mounted on the immobilizing carrier was irradiated with laser beam of linear polarization and of a 488-nm wavelength and a beam diameter of about 3 mm using argon laser of an air cooling type and at an output power of 40 mW for 5 minutes.

After the light irradiation, the area optically irradiated was observed, using a contact-mode atomic force microscope (“Nanoscope E” under trade name as manufactured by Digital Instrument). FIG. 4 shows the results of the observation (observed image). As apparently shown in FIG. 4, the polystyrene microspheres self-assembling (autonomously arranged into a hexagonal structure in this case) and having been immobilized on the surface of the immobilizing carrier was observed.

Then, the immobilizing carrier having immobilized the polystyrene microsphere was immersed in benzene to dissolve the polystyrene microsphere. Due to the property of the material, the immobilizing carrier is never dissolved in benzene. Thereafter, the immobilizing carrier was taken out and dried, and the surface was observed by the atomic force microscope. FIG. 5 shows the results of the observation (observed image). As apparently shown in FIG. 5, dents of the corresponding shapes were formed at positions corresponding to the positions of the polystyrene microsphere arranged and immobilized on the surface of the immobilizing carrier.

### Comparative Example 1

In absolutely the same manner as in Example 1 except for no laser beam irradiation, Comparative Example 1 was done. The polystyrene microsphere arranged on the immobilizing carrier was observed with the atomic force microscope. The appearance that the polystyrene microsphere was arranged at a density not so different from the density in Example 1 could be observed. However, the observed image contained lots of noise, with no recovery of any sharp observed image. After dissolving the polystyrene microsphere in benzene in the same manner as described above, the surface of the immobilizing carrier was observed with the atomic force microscope. Almost no deformation occurring on the surface of the immobilizing carrier was observed.

### Discussion About Example 1 and Comparative Example 1

Taking account of the results of Example 1 and Comparative Example 1, the present inventors considered that the presence or absence of the dents formed correspondingly to the polystyrene microsphere on the surface of the immobilizing carrier might have some relation with the difference in the observed images. In other words, a possibility is sufficiently high, such that the reason of no recovery of sharp observed image in Comparative Example 1 is due to the positional displacement of the polystyrene microsphere during the atomic force microscopic observation. If this is true, the reason of the recovery of such sharp observed image in Example 1 may possibly have a relation with the formation of the dents.

Based on such consideration, the inventors speculated that any formation of dents corresponding to the shape of the polystyrene microsphere on the surface of the immobilizing carrier under light irradiation would distinctly enhance the immobilizing potency of the immobilizing carrier over the polystyrene microsphere. The reason is probably ascribed to the effect of the dents on the support of the polystyrene microsphere, the increase of van der Waals force due to the increase of the area in close contact or the like. The speculation is not essentially affirmed, singly based on the results of Example 1 and Comparative Example 1. So as to verify such immobilizing potency, thus, the following Example 1-2, Example 2 and Comparative Example 2 were carried out.

### Example 1-2

In the same manner as in Example 1, an immobilizing carrier of a thin film was prepared on slide glass. A great number of a polystyrene microsphere of a diameter of 1,000 nm was arranged on the immobilizing carrier.

Then, the area where the polystyrene microsphere was arranged on the immobilizing carrier was irradiated with laser beam for 5 minutes. The laser beam was of a linear beam cross section of a length of 1 cm and a width of about 20 μm, as modified from the laser beam of a beam diameter of about 1 cm and a wavelength of 488 nm from argon laser at an output of 20 mW, using a cylindrical lens (CLB-3030-50 PM manufactured by Sigma Koki Co., Ltd.).

After the light irradiation, the immobilizing carrier together with the slide glass was immersed in water in an ultrasonicator (900-W output) for ultrasonic rinsing. Thereafter, the immobilizing carrier was taken out and dried, for the observation of the area irradiated by the laser beam, using a dark field microscope. The results of the observation with a subject lens of magnification×20 are shown in FIG. 15. As apparently shown in FIG. 15, the polystyrene microspheres attached linearly to the optically irradiated area, depending on the beam cross section of the irradiation beam, while the polystyrene microspheres were almost removed from the surrounding areas. In other words, it was confirmed that optical irradiation on the polystyrene microsphere on the immobilizing carrier strongly immobilized the polystyrene microsphere and never detached the polystyrene microsphere from the surface of the immobilizing carrier, even by the impact of ultrasonic rinsing.

### Example 2

According to the method described in J. Am. Chem. Soc. 121, 961 (1999), an organic-inorganic hybrid mesoporous material in fine particle was prepared. The porous particle was treated in the same manner as for the polystyrene microsphere in Example 1, so that the porous particle was spread and arranged on the same immobilizing carrier as in Example 1. After water evaporation, the area where the porous particle was arranged on the immobilizing carrier was irradiated with laser beam of linear polarization and of a wavelength of 488 nm and a beam diameter of about 3 mm, using argon-krypton laser (10-mW output) for 60 minutes.

After the light irradiation, the immobilizing carrier was immersed in water, for ultrasonic rinsing. The output of the ultrasonicator is 90 W. Thereafter, the immobilizing carrier was taken out and dried, for the observation of the area irradiated with the laser beam, using the atomic force microscope. The results of the observation (observed image) are shown in FIG. 6. FIG. 6 shows the appearance of the porous particle immobilized at a high density on the surface of the immobilizing carrier. This means that the porous particle was never detached from the surface of the immobilizing carrier even via the ultrasonic rinsing in water. Thus, the strong immobilization potency of the porous particle could be confirmed.

FIG. 7 is an enlarged view of a part enclosed with a square frame in white in FIG. 6. Further, FIG. 8 shows an enlarged view of a part enclosed with a square frame in white in FIG. 7. It is suggested that the porous particle is strongly immobilized on the surface of the immobilizing carrier, in the light of the recovery of sharp observed images in the enlarged views in FIG. 7 and FIG. 8.

### Comparative Example 2

In the same manner as in Example 2 except for no irradiation of laser beam, Comparative Example 2 was done. Then, it was attempted to observe the porous particle arranged on the immobilizing carrier with the atomic force microscope. However, the porous particle remained only very sparsely on the surface of the immobilizing carrier. Based on comparison with Comparative Example 1, the reason may be that most of the porous particles were detached due to the insufficient immobilization potency during the ultrasonic rinsing in water. Additionally, it was attempted to observe a few number of the porous particles remaining on the surface of the immobilizing carrier with the atomic force microscope. However, sharp observed images like FIG. 6 to FIG. 8 were absolutely never obtained, because of the large noise.

### Example 3

After a silicon substrate of a size 1 cm×1 cm was treated with a coupler, a polyimide (“PIX” under trade name as manufactured by Hitachi Chemical Co., Ltd. was used) film of a film thickness of 7 μm was formed thereon by spin coat method, and was then heated at a preset temperature. Using the polymer compound of the formula 6, a thin film of a thickness of about 500 μm was further prepared on the polyimide film, to obtain an immobilizing carrier substrate. The thin film was prepared as follows. A solution of the polymer compound of the formula 6 dissolved in pyridine to 6.5% by weight was prepared and filtered through a 0.2-μm filter. Subsequently, the resulting filtrate was spin coated on slide glass at a rotation number of 1,000 rpm, and vacuum dried at 80° C. for 20 hours and then vacuum dried at 150° C. for 2 hours, to prepare the thin film.

10 mg of a protease [PROTEASE (Subtillisin Carlsberg)] was dissolved in 10 mL of ion exchange water. At the state of the immobilizing carrier substrate immersed in the resulting aqueous enzyme solution, the face of the immobilizing carrier substrate was irradiated with laser beam of a wavelength of 488 nm and an intensity of 80 mW/cm² for 30 minutes. In Comparative Example 3 corresponding to Example 3, the immobilizing carrier substrate was immersed in the aqueous enzyme solution for 30 minutes, without any laser beam irradiation. These immobilizing carrier substrates of Example 3 and Comparative Example 3 were taken out from the aqueous enzyme solutions and were then immersed separately in ion exchange water for rinsing.

On the other hand, 4 mg of an artificial substrate (BOC-GGL-PNA) was dissolved in 1 mL of dimethylformamide. When the artificial substrate is decomposed with protease, p-nitroanilide is solubilized therefrom, involving the increase of the absorption at a wavelength of 380 nm. Thus, the solution is colored yellow. 50 μl of the artificial substrate solution was taken out and added to 450 μl of 10 mM Tris-HCl buffer, pH 8.0, to prepare a solution for reaction.

50 μl each of the solution for reaction was dropwise added onto the surface of each of the immobilizing carrier substrates of Example 3 and Comparative Example 3. The resulting carrier substrates were left to stand at 37° C. for one hour. Thereafter, the individual solutions for reaction were recovered under aspiration and were measured of the absorbance at 380 nm, using UV-VIS spectrometer. Consequently, the absorbance of the solution for reaction as dropwise added onto the immobilizing carrier substrate of Example 3 increased by 0.117, while the absorbance of the solution for reaction as dropwise added onto the immobilizing carrier substrate of Comparative Example 3 increased by 0.018.

Because any of the immobilizing carrier substrates was immersed in the aqueous enzyme solution and then rinsed with ion exchange water, as described above, it is indicated that the difference in the increment of the absorbance (the difference in the enzyme activity) reflects the difference in the immobilization potency of the enzyme between the immobilizing carrier substrates and that the difference in the immobilizing potency may be ascribed to the presence or absence of the irradiation of laser beam.

Using the atomic force microscope, the shapes of the surfaces of the immobilizing carrier substrates of Example 3 and Comparative Example 3 were observed. The results are shown in FIG. 9 (Example 3) and FIG. 10 (Comparative Example 3). In FIG. 9, recesses and protrusions of sizes of about 5 nm to about 20 nm were observed. In FIG. 10, recesses and protrusions of about 1 to 2 nm or less were only observed. The recesses and protrusions of sizes of 5 to 20 nm in FIG. 9 show the enzyme immobilized on the surface of the immobilizing carrier substrate.

### Example 4

After a silicon substrate of a size 1 cm×1 cm was treated with a coupler, a polyimide (“PIX” under trade name as manufactured by Hitachi Chemical Co., Ltd. was used) film of a film thickness of 7 μm was formed thereon by spin coat method, and was then heated at a preset temperature. Using the polymer compound of the formula 6, a thin film of a thickness of about 500 μm was further prepared on the polyimide film, to obtain an immobilizing carrier substrate. The thin film was prepared as follows. A solution of the polymer compound of the formula 6 dissolved in pyridine to 6.5% by weight was prepared and filtered through a 0.2-μm filter. Subsequently, the resulting filtrate was spin coated on slide glass at a rotation number of 1,000 rpm, and vacuum dried at 80° C. for 20 hours and then vacuum dried at 150° C. for 2 hours, to prepare the thin film.

λ-DNA (48,502 bp; manufactured by Nippon Gene, Co., Ltd.) was dissolved in TE buffer, pH 8.0 to 5.5 μM (base). After 10 μl of the solution was dropwise added onto the immobilizing carrier substrate, the immobilizing carrier substrate was rotated at 1,500 rpm, to spin cast λ-DNA on the immobilizing carrier substrate. Then, the immobilizing carrier substrate was irradiated with laser beam of a wavelength of 488 nm and an intensity of 80 mW/cm² for 30 minutes. In Comparative Example 4 corresponding to Example 4, λ-DNA was spin cast on the immobilizing carrier substrate in the same manner, without any laser beam irradiation.

Using the atomic force microscope, the shapes of the surfaces of the immobilizing carrier substrates of Example 4 and Comparative Example 4 were observed individually in duplicate. FIG. 11 shows the first scanned image of Example 4 and FIG. 12 shows the second scanned image thereof. FIG. 13 shows the first scanned image of Comparative Example 4 and FIG. 14 shows the second scanned image thereof. In FIG. 11, the linear molecule of λ-DNA could be observed sharply in the center of the figure. Even in FIG. 12 as the second scanned image, no change in the linear molecular image of λ-DNA was observed. In FIG. 13 of Comparative Example 4, the linear molecule of λ-DNA could be observed, with more noises compared with the noise in FIG. 11. In FIG. 14 as the second scanned image, the linear molecular image of λ-DNA changed. It is considered as the reason that because λ-DNA was not sufficiently immobilized on the surface of the immobilizing carrier substrate in Comparative Example 4, λ-DNA displaced because of the scanning of the atomic force microscope of contact mode.

### Example 5

(Example 5-1: Material for optical immobilization, containing a dye structure with amino group and cyano group)

Using the known diazo coupling process, the azo dye compound shown by the formula 7 was synthetically prepared.

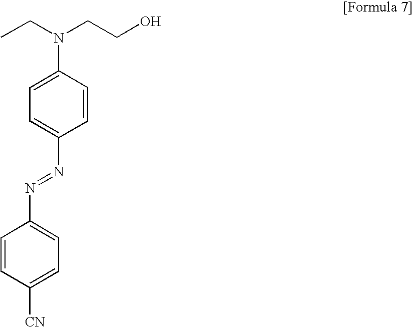

Specifically, 5.9 g of 4-aminobenzonitrile was added to 100 mL of ion exchange water and 45 mL of 36% hydrochloric acid while they were being mixed together with stirring in a 500-mL beaker. Then, 18 mL of a solution of 3.9 g of sodium nitrite dissolved in water was gradually dropwise added over 15 minutes. After stirring continued as it was for 30 minutes, 125 mL of a solution of 8.3 g of 2-(N-ethylanilino)ethanol, 7.5 mL of 36% hydrochloric acid and 125 mL of ion exchange water mixed together and stirred together was dropwise added over 30 minutes. The reactions so far were all carried out under ice-cold conditions.

After the reaction mixture was back to ambient temperature, 200 mL of water dissolving 35.4 g of potassium hydroxide therein was added in portions to the reaction solution, to recover the red precipitate in deposition. The red precipitate was recrystallized five times in ethanol, to obtain the red crystal of the azo dye compound shown by the formula 7. The crystal was examined by TLC with a mix solution of ethyl acetate:hexane (1:1). There was only one spot. The yield was 0.65.

Then, the azo dye compound shown by the formula 7 reacted with an acid chloride for methacrylation, to synthetically prepare the compound shown by the formula 8.

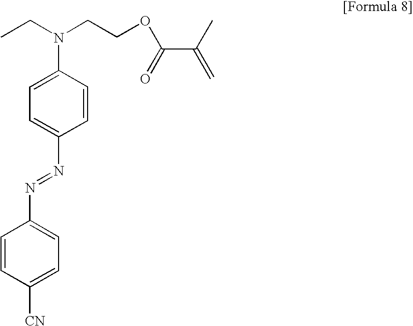

Specifically, 3 g of the azo dye compound shown by the formula 7, 1 g of pyridine and 10 mL of tetrahydrofuran (THF) were placed and agitated together in a 100-mL round-bottom flask. Cooling then the round-bottom flask in ice, 10 mL of THF dissolving 1.5 g of methacryloyl chloride therein was dropwise added into the round-bottom flask. Continuing stirring as it was for 30 minutes, the generation of pyridinium salt was confirmed. The round-bottom flask was back to ambient temperature. The reaction solution was filtered to discard the pyridinium salt. After the filtrate was distilled under reduced pressure, the resulting product was purified by column chromatography (ethyl acetate/hexane=1:1). The yield was 0.71. Using 1H-NMR, 13C-NMR and infrared absorption spectrum, the synthesis of the compound shown by the formula 8 was verified.

A material for optical immobilization was synthetically prepared by the copolymerization of the compound shown by the formula 8 with methyl methacrylate (MMA). Specifically, 0.362 g of the compound shown by the formula 8 and 0.9 g of MMA from which polymerization inhibitors have preliminarily been removed by distillation under reduced pressure were mixed together in a 100-mL round bottom flask, followed by addition of 50 mL of dimethylformamide and 82 mg of 2,2-azoisobutylonitrile. Closing the round-bottom flask with a rubber stopper as the lid, oxygen was removed from the system in nitrogen bubbling for one hour. Still in nitrogen bubbling, then, the round-bottom flask was heated to 60° C. Two hours later, the reaction solution was taken out from the round-bottom flask, for reprecipitation with methanol. The reprecipitation was repeated three times. After subsequent drying under reduced pressure, a material for optical immobilization was obtained as a polymer material prepared by the copolymerization of the compound shown by the formula 8 with methyl methacrylate (MMA).

(Example 5-2: Material for optical immobilization, containing a dye structure with amino group and nitro group)

The present Example is a comparative example corresponding to Example 5-1. Using Disperse Red 1 (DR1) of the formula 9 as a commercially available azo dye compound instead of the azo dye compound shown by the formula 7, the azo dye was acrylated via reaction with acid chloride in the same manner as in Example 5-1, to synthetically prepare the compound shown by the formula 10. Subsequent copolymerization of the compound shown by the formula 10 with methyl methacrylate (MMA) in the same manner as in Example 5-1 synthetically prepared a material for optical immobilization as a polymer material.

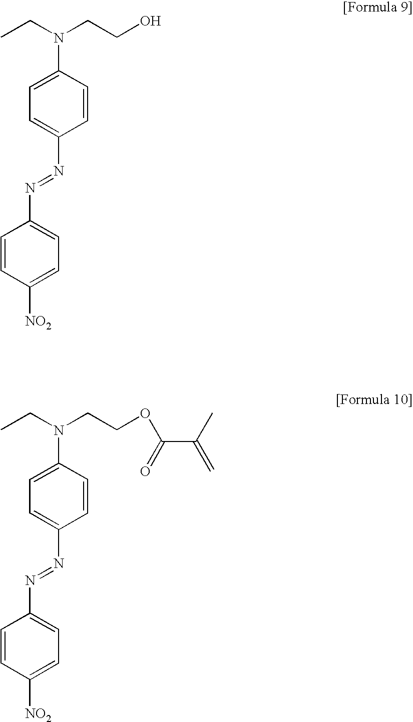

(Example 5-3: Preparation of polymer film and ultraviolet and visible absorption spectra)

50 mg of the material for optical immobilization as synthetically prepared in Example 5-1 and 50 mg of the material for optical immobilization as synthetically prepared in Example 5-2 were individually dissolved in 2 mL of pyridine. These solutions were dropwise added in each 1-mL portion onto slide glass substrates, from which the solvents were removed via the rotation of the slide glass substrates at 2,000 rpm, to prepare polymer films of a uniform thickness. The film thickness of any of the resulting films was about 110 nm.

The ultraviolet and visible absorption spectra of these polymer films were measured, using UV-VIS spectrophotometers. The results are shown in FIG. 16. FIG. 16 indicates that the absorption band of the spectral chart (expressed in solid line) of the polymer film of Example 5-1 was in the region of shorter wavelength, compared with that of the spectral chart expressed in dotted line of the polymer film of Example 5-2. The spectral chart expressed in solid line has the 570-nm cut-off wavelength of the absorption band in the region of longer wavelength, which is in the region of shorter wavelength than the fluorescence peak wavelength of the fluorescence dye Cy3 shown concurrently in the figure. On the other hand, the spectral line expressed in dotted line has the 650-nmm cut-off wavelength of the absorption band in the region of longer wavelength, which is in the region of longer wavelength than the fluorescence peak wavelength of the fluorescence dye Cy3.

(Example 5-4: Preparation of DNA microarray and fluorescence detection)

Two 1-mL Eppendorf tubes placing 100 μL of a solution of λ-DNA (Nippon Gene, Co., Ltd.; 48 kbp; 0.5 μg/μL) in each of the tubes were prepared. 1 μL each of the stock solutions of fluorescence dyes (PO-PRO-3 iodide and TO-PRO-3 iodide manufactured by Molecular Probe Co., Ltd.) for DNA staining were added separately into these individual Eppendorf tubes and agitated. Herein, PO-PRO-3 iodide emits fluorescence with a peak at 570 nm almost similar to the fluorescence wavelength of Cy3, while TO-PRO-3 iodide emits fluorescence with a peak at 670 nm almost similar to the fluorescence wavelength of Cy5.

The λ-DNA's thus stained with the fluorescence dyes were spotted on the polymer film of Example 5-1 and the polymer film of Example 5-2, using 417 Arrayer manufactured by Affimetrix Co., Ltd. Spotting followed the design of FIG. 17. In FIG. 17, lane 1 shows the CNA spot stained with PO-PRO-3 iodide and lane 2 shows the CNA spot stained with TO-PRO-3 iodide. Actually the diameter of each spot was 125 μm, while the spot interval was 375 μm.

The fluorescence from the λ-DNA's spotted on the polymer films was analyzed using 428 Array Scanner manufactured by Affimetrix Co., Ltd. Among the results of the analysis using a filter set (excitation at 532 nm and fluorescence at 560-580 nm) for Cy3, FIG. 18A shows the results of the polymer film of Example 5-1 and FIG. 18B shows the results of the polymer film of Example 5-2. Among the results of the analysis using a filter set (excitation at 632 nm and fluorescence at 660-680 nm) for Cy5, FIG. 19A shows the results of the polymer film of Example 5-1 and FIG. 19B shows the results of the polymer film of Example 5-2.

Based on these comparisons, the results of the polymer film of Example 5-1 as shown in FIG. 18A and FIG. 19A have better contrasts than those of the results of the polymer film of Example 5-2 as shown in FIG. 18B and FIG. 19B. As described above, fluorescence analysis using fluorescence dyes corresponding to Cy3 and Cy5 can be satisfactorily done, in case that the cut-off wavelength of a dye contained in the polymer material in the region of longer wavelength is 570 nm or less.

### Example 6

### Biosensor

### Example 6-1: Antigen-Antibody Reaction and Detection of the Fluorescence

The material for optical immobilization as a polymer prepared in Example 5-2 was dissolved in pyridine. Several hundreds milliliters of the resulting solution were dropwise added onto slide glass. Subsequently, the slide glass was rotated at a rotation number of 1,000 rpm, to prepare a thin film of a film thickness of about 1 μm on the slide glass by the spin coat method. This was used as a carrier for immobilization for the following procedures.

As ligands, bovine serum albumin (BSA) and human serum albumin (HSA) were individually dissolved in a buffer to concentrations of 10, 5, 1, and 0.5 (μg/μl). Subsequently, two drops of each of the resulting buffers (1 μl per one spot) were dropwise added onto the carrier in separate regions, using pipettes. FIG. 21 shows the layout of the spots. Then, using a green LED light source (at a light intensity of about 5 mW/cm²), light irradiation toward the carrier surface was done for one hour, to immobilize BSA and HSA.

So as to cover the BSA-immobilized region and the HSA-immobilized region on the carrier, gap cover glass (manufactured by Matsunami Glass) was put on the carrier surface. Mouse anti-HSA monoclonal antibodies dissolved in a phosphate buffer to a concentration of 0.001 μg/μl were infiltrated into the gap between the gap cover glass and the carrier surface. 30 minutes later, the carrier together with the gap cover glass was immersed in the phosphate buffer, from which the gap cover glass was then removed. Then, the carrier was once again immersed in another phosphate buffer, for rinsing under agitation.

So as to cover the BSA-immobilized region and the HSA-immobilized region on the carrier, once again, gap cover glass (manufactured by Matsunami Glass) was put on the carrier surface. Goat anti-mouse antibodies labeled with a fluorescence substance Cy5 as dissolved in a phosphate buffer to a concentration of 0.001 μg/μl were infiltrated into the gap between the gap cover glass and the carrier surface. 30 minutes later, the carrier together with the gap cover glass was immersed in the phosphate buffer, from which the gap cover glass was removed. Then, the carrier was once again immersed in another phosphate buffer, for rinsing under agitation.

At that state, the surface of the carrier was observed, using a fluorescence microscope. The emission of fluorescence from the HSA-immobilized region was confirmed. However, no emission of fluorescence from the BSA-immobilized region was confirmed. FIG. 22 shows these fluorescence microscopic images. The layout of the spots in FIG. 22 is the same as in FIG. 21, indicating that only the HSA-immobilized region emits fluorescence.

So as to cover the BSA-immobilized region and the HSA-immobilized region on the carrier, further, gap cover glass (manufactured by Matsunami Glass) was put on the carrier surface. An aqueous 10 mM hydrochloric acid solution was infiltrated into the gap between the gap cover glass and the carrier surface. 30 minutes later, then, the carrier together with the gap cover glass was immersed in ion exchange -water, from which the gap cover glass was removed.

At that state, the surface of the carrier was observed, using a fluorescence microscope. The emission of fluorescence from the HSA-immobilized region or from the BSA-immobilized region was never confirmed. In other words, at least the absence of the goat anti-mouse antibodies labeled with Cy5 on the carrier were confirmed. FIG. 23 shows these fluorescence microscopic images. The layout of the spots in FIG. 22 is the same as in FIG. 21, indicating that neither the HSA-immobilized region nor the BSA-immobilized region emits fluorescence.

Once again, the HSA/BSA-immobilized carrier at that state was treated with the treatment using the mouse anti-HSA monoclonal antibody and with the treatment using the goat anti-mouse antibody labeled with Cy5. Then, the surface of the carrier was observed, using a fluorescence microscope. The emission of fluorescence from the HSA-immobilized region was confirmed, but no fluorescence from the BSA-immobilized region was observed. FIG. 24 shows these fluorescence microscopic images. The layout of the spots in FIG. 24 is the same as in FIG. 21, indicating that only the HSA-immobilized region emits fluorescence, as in the case of FIG. 22.

The above results indicate those described below. 1) The method for optical immobilization can immobilize antigen on the carrier of the invention. 2) Using the antigen immobilized on the carrier, antigen-antibody reaction can be progressed. In other words, the antigen is immobilized as it remains in the active type structure. 3) Using fluorescence, the antigen-antibody reaction can be detected. 4) In case of the optical immobilization of the invention, no adsorption of antibody occurs at sites except for the site with the spotted antigen, even without any specific blocking treatment of the carrier. 5) Treatment with hydrochloric acid after antigen-antibody reaction detaches only the antibody, while the optically immobilized antigen is never detached from the carrier surface or is never inactivated.

(Example 6-2: Optical patterning of protein)

The material for optical immobilization as a polymer prepared in Example 5-2 was dissolved in pyridine. Several hundreds milliliters of the resulting solution were dropwise added onto slide glass. Subsequently, the slide glass was rotated at a rotation number of 1,000 rpm, to prepare a thin film of a film thickness of about 1 μm on the slide glass by the spin coat method. This was used as a carrier for immobilization, for the following procedures.

Gap cover glass (manufactured by Matsunami Glass) was put on the surface of the carrier. Concurrently, one triangle-shaped aluminium sheet piece was allowed to closely adhere to the surface of the gap cover glass, to arrange a region with no transmission of light. Then, a phosphate buffer dissolving HSA to a concentration of 0.001 μg/μl therein was infiltrated into the gap between the gap cover glass and the carrier surface. Using a green LED light source (at a light intensity of about 5 mW/cm²), light irradiation toward the carrier surface was done for one hour. Then, the carrier together with the gap cover glass was immersed in the phosphate buffer, from which the gap cover glass was removed. Then, the carrier was once again immersed in another phosphate buffer, and was rinsed under agitation therein.

So as to cover the region where the light irradiation was done on the carrier, the gap cover glass was put on the carrier. A phosphate buffer dissolving the mouse anti-HSA monoclonal antibodies to a concentration of 0.001 μg/μl therein was infiltrated into the gap between the gap cover glass and the carrier surface. 30 minutes later, the carrier together with the gap cover glass was immersed in the phosphate buffer, from which the gap cover glass was removed. Then, the carrier was once again immersed in another phosphate buffer, for rinsing under agitation.

Once again, the gap cover glass was put on the surface of the carrier, so as to cover the region where the light irradiation was done on the carrier. Then, a phosphate buffer dissolving goat anti-mouse monoclonal antibodies labeled with a fluorescence substance Cy5 to a concentration of 0.001 μg/μl therein was infiltrated into the gap. 30 minutes later, then, the carrier was once again immersed in another phosphate buffer, from which the gap cover glass was removed. The carrier was again immersed in another phosphate buffer, for rinsing under agitation therein.

At that state, the surface of the carrier was observed, using a fluorescence microscope. It was confirmed that the region within the range where the light irradiation was done on the carrier, except for the light-non-transmitting region set by the aluminium sheet piece, emitted fluorescence. FIG. 25 shows the fluorescence microscopic image. In FIG. 25, the sharp triangle region with no emission of fluorescence, which corresponds to the aluminium sheet piece, can be observed. By introducing a specific pattern in a region to be optically irradiated in such manner, protein patterning via optical immobilization is enabled.

(Example 6-3: Biosensor of SPR mode)

Biosensor carrier 13 for the SPR test method was fabricated, as shown in FIG. 20. Specifically, metal film 15 prepared by depositing metal to a film thickness of 50 nm was prepared on glass substrate 14 as slide glass. Additionally thereon, thin film 16 of a material for optical immobilization, which was of a film thickness of about 20 nm, was prepared by the spin coat method using the pyridine solution of the same polymer compound 2 as in Example 6-1.

After subsequently dropwise adding matching oil on prism 17, the biosensor carrier 13 was left to stand thereon, while the side of the glass substrate 14 was faced underneath. So as to prepare total reflection conditions in the interface between the deposited gold and the glass substrate, the interface was irradiated with laser beam of a wavelength of 633 nm from He-Ne laser source 18. Using photodiode 19, further, the laser beam reflected was detected. The angle of the photodiode 19 to the laser was controlled, using a goniometer. Then, the gap cover glass was put on the surface of the biosensor carrier 13. A buffer flowed in the gap between the gap cover glass and the carrier surface. At the state, SPR signal was detected. Compared with the measurement using only the thin gold film without any deposition on the biosensor 13, the SPR angle was shifted by about 10 degrees to the side of wider angle.

The gap cover glass was put on the surface of the biosensor carrier 13 thus fabricated. An aqueous solution of HSA for use as ligand was prepared to a concentration of 5 μg/μL. The solution was infiltrated into the gap between the gap cover glass and the carrier surface. Using a green LED light source (at a light intensity of about 5 mW/cm²), light irradiation toward the carrier surface was done at that state for one hour, to immobilize HSA. Then, the carrier together with the gap cover glass was immersed in a phosphate buffer, from which the gap cover glass was removed. Then, the carrier was once again immersed in another phosphate buffer, and was rinsed under agitation therein. On the other hand, an aqueous solution of BSA for use as ligand was also prepared at the same concentration. In the same manner, BSA was immobilized.

The biosensor carriers 13 immobilizing thereon HSA and BSA were left to stand on the prism 17, to which matching oil was preliminarily dropwise added in the same manner as described above. To prepare total reflection conditions in the interface between the deposited gold and the glass substrate, the interface was irradiated with laser beam of a wavelength of 633 nm from the He-Ne laser source 18. Using photodiode 19, further, the laser beam reflected was detected. The angle of the photodiode 19 to the laser was controlled, using a goniometer.

Then, the gap cover glass was put on the surface of the biosensor carrier having immobilized HSA and BSA thereon, so as to cover the individual immobilized regions. An aqueous solution of a mouse anti-HSA monoclonal antibody was infiltrated between the gaps of the gap cover glass. At the state, SPR signal was detected. The SPR angle on the HSA-immobilized biosensor carrier where the binding of the ligand and the antibody was expected was gradually shifted toward the wider angle side. However, no change of the SPR angle was observed on the BSA-immobilized biosensor carrier.

### Example 7

### Electrochemical Biosensor

A polymer compound (azopolymer-1) shown by the formula 11 and a polymer compound (azopolymer-2) shown by the formula 6 were synthetically prepared.

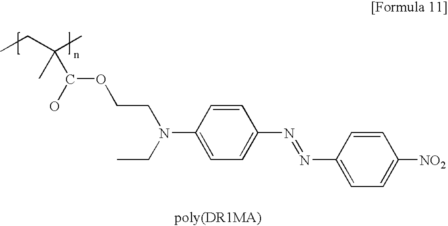

Azopolymer-1 was dissolved in NMP to a given concentration and was then coated on glass substrate by the spin coat method, to prepare an immobilizing material layer. Platinum was deposited on the surface of the immobilizing material layer, to prepare a pair of electrodes. The electrode on one side was furthermore silver plated. Then, a phosphate buffer solution of glucose oxidase was spotted in the middle between the pair of electrodes by the spotting method. From the above, laser beam from argon laser of a wavelength of 488 nm irradiated for 30 minutes.

The enzyme electrodes thus prepared were connected to a potentiostat and were then immersed in a sodium chloride solution containing glucose, to which voltage was applied. Electric current based on hydrogen peroxide generated via the reaction between glucose oxidase and glucose could be observed.

By the same method as described above, subsequently, enzyme electrodes with immobilized L-ascorbate oxidase were prepared. Then, the electrodes were immersed in an L-ascorbate solution, followed by the application of voltage. Then, an electric current could be observed.

Using the azopolymer-2, further, the glucose oxidase-immobilized enzyme electrodes and the L-ascorbate-oxidase-immobilized enzyme electrodes as described above were prepared. The electrodes were then immersed in the given substrate solutions, to which voltage was applied. Electric current could be observed with any of the electrodes.

The above results indicate that the invention can immobilize enzymes at their active types and can satisfactorily prepare electrochemical biosensors.

While the preferred embodiments have been described, variations thereto will occur to those skilled in the art within the scope of the present inventive concepts, which are delineated by the following claims.

What is claimed is:

1. A method for optically immobilizing very small objects, comprising a step of fabricating a carrier using the following material (A) for optical immobilization at least as a surface layer thereof, a step of arranging the following very small object (B) on the surface of the carrier, and a step of immobilizing the very small objects thus arranged on the surface of the carrier via light irradiation:
(A) the material for optical immobilization: a material with the capability of photoinduced deformation, which exerts the potency of immobilizing the very small objects arranged on the surface of the carrier during light irradiation; and
(B) the very small objects: a tangible object with a size of 50 μm or less.

2. The method according to claim 1, wherein the material for optical immobilization is a material containing a dye structure with azo group.

3. The method according to claim 2, wherein the dye structure with azo group is the azobenzene structure having an aromatic ring containing one or more electron donating substituents with negative values of the substituent constant σ according to the Hammet's rule and an aromatic ring containing one or more electron withdrawing substituents with positive values of the substituent constant σ according to the Hammet's rule, individually on both sides of the azo group.

4. The method according to claim 3, wherein the dye structure with azo group is a dye structure under control so that the cut-off wavelength of the photoabsorption wavelength on the side of longer wavelength may exist in the region of shorter wavelength than the fluorescence peak wavelength of a fluorescence dye for fluorescence analysis, provided that the dye structure has the electron withdrawing substituents and the electron donating substituents under conditions that the following formula 1 can be established:
Σ|σ|≦σ1|+|σ2| [Formula 1]

5. The method according to claim 1, wherein the very small object is one or more selected from the group consisting of (1) to (5):
(1) an inorganic material particle group, including at least metal particles, metal oxide particles, semiconductor particles and ceramic particles;
(2) an organic material particle group, including at least plastic particles;
(3) a high-molecular-weight organic molecule group, including at least polypeptide molecules in chain, protein molecules with active-site or inactive-site, assemblies of these protein molecules, single-stranded or double-stranded or higher-stranded nucleic acid molecules, or polysaccharide molecules;
(4) fine particles of inorganic materials or organic materials, to which high-molecular-weight organic molecules are preliminarily bound; and
(5) cells, organellas, bacteria, viruses, biological tissues or biological organisms, including at least cells, bacteria or biological organisms at viable states thereof in the group.

6. The method according to claim 1, where the arrangement and immobilization of very small objects on the surface of the carrier is carried out in a liquid medium dissolving or suspending the very small objects therein.

7. The method according to claim 1, wherein laser trapping is used for the arrangement of very small objects on the surface of the carrier.

8. The method according to claim 1, wherein a great number of one or more types of very small objects are immobilized following specific distribution patterns differing from each other on the surface of the carrier, by giving preset distributions to the irradiation region or irradiation intensity of the irradiation light.

9. The method according to claim 1, wherein the irradiation light is propagating light, optical near field or evanescent field.

10. A very small object-immobilized carrier having immobilized very small objects on the surface of the carrier by the method for optically immobilizing very small objects according to claim 1.

11. The very small object-immobilized carrier according to claim 10, wherein the very small object-immobilized carrier is an integrated circuit chip where very small objects as either (1) or (2) according to claim 5 are immobilized on an integrated circuit substrate as the carrier, following a preset distribution pattern.

12. The very small object-immobilized carrier according to claim 10, wherein the very small object-immobilized carrier is the following (6) or (7):
(6) a bioreactor or biosensor prepared by immobilizing single species or plural species of enzymes, antibodies, antigens, microorganisms, or organellas as very small objects on the carrier as a reaction bed or substrate; or
(7) a bioassay test piece or a protein chip for proteome analysis, as prepared by immobilizing a protein to be expressed in a biological cell.

13. The very small object-immobilized carrier according to claim 12, wherein the bioreactor or biosensor (6) is a very small object-immobilized carrier having immobilized the very small object in (6) above on the surface of the carrier using the material for optical immobilization at least as a surface layer and having formed electrodes on the surface of the carrier.

14. The very small object-immobilized carrier according to claim 12, wherein the bioassay test piece or the protein chip for proteome analysis as (7) is a very small object-immobilized carrier having formed the film of a material for optical immobilization on the surface of a metal thin film leading to surface plasmon resonance phenomenon, where the protein as a very small object has been immobilized on the surface of the carrier.

15. The very small object-immobilized carrier according to claim 10, wherein the very small object-immobilized carrier is any one of the following (8) to (10):
(8) a DNA chip or DNA microarray immobilizing DNA fragments usable as a genetic marker thereon;
(9) a DNA chip or DNA microarray immobilizing DNA fragments including DNA fragments containing single nucleotide polymorphism (SNP), restriction fragments or DNA fragments containing microsatellite part thereon; and
(10) a DNA chip or DNA microarray immobilizing mRNA or fragments thereof, cDNA or fragments thereof, or fragments of genome DNA thereon.

16. A very small object-immobilized carrier having immobilized very small objects on the surface of a carrier by the method for optically immobilizing very small objects according to claim 2.

17. A very small object-immobilized carrier having immobilized very small objects on the surface of a carrier by the method for optically immobilizing very small objects according to claim 3.

18. A method for observing very small objects comprising a step of immobilizing very small object on the surface of a carrier by the method for optically immobilizing very small objects according to claim 1, and a step of observing the very small objects immobilized by an appropriate process of giving displacement force to the very small objects.

19. The method according to claim 18, wherein the very small objects are cells or microorganisms and the very small objects are observed at the viable state thereof while they remain immobilized.

20. The method according to claim 18, wherein the very small objects are enzymes, antigens, antibodies or cell membrane receptors as polypeptide and the method includes the use of scanning probe microscope as an observation tool to modify the probe with enzyme substrates, antibodies, antigens or cell membrane receptor ligands to thereby observe the reactive part of the enzymes, the antigens, the antibodies or the cell membrane receptors, functionally or in terms of steric configuration.
